(19) United States
(12) Patent Application Publication
(10) Pub. No.: US 2024/0216443 A1
(43) Pub. Date: Jul. 4, 2024

(54) A MYCOBACTERIUM FOR USE IN CANCER THERAPY

(71) Applicant: IMMODULON THERAPEUTICS LIMITED, LONDON (GB)

(72) Inventors: CASPER HENRICUS JOHANNES VAN EIJCK, ROTTERDAM (NL); ANDREW GAYA, LONDON (GB); GLEN MARTYN, LEICESTERSHIRE (GB)

(21) Appl. No.: 18/291,950

(22) PCT Filed: Jul. 27, 2021
(86) PCT No.: PCT/GB2021/051935

§ 371 Date: Jan. 25, 2024

(51) Int. Cl. A61K 35/74; A61K 45/06; A61N 5/10; A61P 35/04

(52) U.S. Cl. CPC A61K 35/74; A61K 45/06; A61N 5/10; A61P 35/04

(57) ABSTRACT

This invention provides an immunomodulator for use in the treatment or control of cancer comprising a primary tumour, in a patient that has undergone, or is intended to undergo tumour resection surgery, comprising simultaneously, separately, or sequentially administering one or more doses of ionizing radiation selected from photon therapy or particle therapy to the patient, wherein said immunomodulator comprises non-pathogenic non-viable Mycobacterium. The invention also describes the use of one or more additional anticancer treatments or agents as well as protocols and dosage regimens for use.

### FIELD OF THE INVENTION

The present invention relates to the field of cancer therapy. In particular, the present invention relates to the treatment or control of cancer in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, in combination with a Mycobacterium preparation.

### BACKGROUND OF THE INVENTION

In humans with cancer, treatment regimens often include forms of oncological surgical intervention, chemo-, radio- or immuno-therapies. In advanced cancer, anti-tumour immunity is often ineffective due to the tightly regulated interplay of pro- and anti-inflammatory, immune-stimulatory and immunosuppressive signals. For example, loss of the anti-inflammatory signals leads to chronic inflammation and prolonged proliferative signalling. Interestingly, cytokines that both promote and suppress proliferation of the tumour cells are produced at the tumour site. It is the imbalance between the effects of these various processes that results in tumour promotion.

Pancreatic cancer is the third most common cause of cancer death. The mean life expectancy from diagnosis to death is approximately 6 to 12 months. Unfortunately, the low survival rate is due to the lack of symptoms in the early stages of cancer development. Therefore, by the time symptoms manifest, the pancreatic cancer has already started to metastasise making pancreatic cancer difficult to treat successfully.

To date, a major barrier to attempts to develop effective immunotherapy for cancers, such as pancreatic cancer, has been an inability to break immunosuppression at the cancer site and restore normal networks of immune reactivity. The physiological approach of immunotherapy is to normalize the immune reactivity so that, for example, the endogenous tumour antigens would be recognized and effective cytolytic responses would be developed against tumour cells. Although it was once unclear if tumour immunosurveillance existed, it is now believed that the immune system constantly monitors and eliminates newly transformed cells. Accordingly, cancer cells may alter their phenotype in response to immune pressure in order to escape attack (immunoediting) and upregulate expression of inhibitory signals. Through immunoediting and other subversive processes, primary tumours and metastasis maintain their own survival. Metastatic cancer cells leave the tumour as microcolonies, containing lymphocytes and platelets as well as tumour cells and may deposit in a tissue as micrometastases. Inflammation continues to play a role at metastatic sites by creating a cytokine milieu conducive to tumour growth.

One of the major mechanisms of anti-tumour immunity subversion is known as T-cell exhaustion, which results from chronic exposure to antigens and is characterized by the up-regulation of inhibitory receptors. These inhibitory receptors serve as immune checkpoints in order to prevent uncontrolled immune reactions. PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (LAG-3; CD223), and others are often referred to as checkpoint regulators. They act as molecular “tollbooths,” which allow extracellular information to dictate whether cell cycle progression and other intracellular signalling processes should proceed.

In addition to specific antigen recognition through the T-cell Receptor (TCR), T-cell activation is regulated through a balance of positive and negative signals provided by costimulatory receptors. These surface proteins are typically members of either the TNF receptor or B7 superfamilies. Agonistic antibodies directed against activating co-stimulatory molecules and blocking antibodies against negative costimulatory molecules may enhance T-cell stimulation to promote tumour destruction. Programmed Cell Death Protein 1, (PD-1 or CD279), a 55-kD type 1 transmembrane protein, is a member of the CD28 family of T cell co-stimulatory receptors that include immunoglobulin superfamily member CD28, CTLA-4, inducible co-stimulator (ICOS), and BTLA. PD-1 is highly expressed on activated T cells and B cells. PD-1 expression can also be detected on memory T-cell subsets with variable levels of expression.

Two ligands specific for PD-1 have been identified: programmed death-ligand 1 (PD-L1), also known as B7-H1 or CD274) and PD-L2 (also known as B7-DC or CD273). PD-L1 and PD-L2 have been shown to down-regulate T cell activation upon binding to PD-1 in both mouse and human systems. The interaction of PD-1 with its ligands, PD-L1 and PD-L2, which are expressed on antigen-presenting cells (APCs) and dendritic cells (DCs), transmits negative regulatory stimuli to down-modulate the activated T cell immune response. Blockade of PD-1 suppresses this negative signal and amplifies T cell responses.

Numerous studies indicate that the cancer microenvironment manipulates the PD-L1-/PD-1 signalling pathway and that induction of PD-L1 expression is associated with inhibition of immune responses against cancer, such as pancreatic cancer, thus permitting cancer progression and metastasis. The PD-L1/PD-1 signalling pathway is a primary mechanism of cancer immune evasion for several reasons. First, and most importantly, this pathway is involved in negative regulation of immune responses of activated T effector cells, found in the periphery. Second, PD-L1 is up-regulated in cancer microenvironments, while PD-1 is also up-regulated on activated tumour infiltrating T cells, thus possibly potentiating a vicious cycle of inhibition. Third, this pathway is intricately involved in both innate and adaptive immune regulation through bi-directional signalling. These factors make the PD-1/PD-L1 complex a central point through which cancer can manipulate immune responses and promote its own progression.

The first immune-checkpoint inhibitor to be tested in a clinical trial was ipilimumab (Yervoy, Bristol-Myers Squibb), a CTLA-4 mAb. CTLA-4 belongs to the immunoglobulin superfamily of receptors, which also includes PD-1, BTLA, TIM-3, and V-domain immunoglobulin suppressor of T cell activation (VISTA). Anti-CTLA-4 mAb is a powerful checkpoint inhibitor which removes “the break” from both naive and antigen-experienced cells. Therapy enhances the antitumor function of CD8+ T cells, increases the ratio of CD8+ T cells to Foxp3+ T regulatory cells, and inhibits the suppressive function of T regulatory cells. The major drawback to anti-CTLA-4 mAb therapy is the generation of autoimmune toxicities due to on-target effects of an over-exuberant immune system which has lost the ability to turn itself down. It has been reported that up to 25% of patients treated with ipilimumab developed serious grade 3-4 adverse events/autoimmune-type side effects including dermatitis, enterocolitis, hepatitis, endocrinopathies (including hypophysitis, thyroiditis, and adrenalitis), arthritis, uveitis, nephritis, and aseptic meningitis. In contrast to the anti-CTLA-4 experience, anti-PD-1 therapy appears to be better-tolerated and induces a relatively lower rate of autoimmune-type side effects.

TIM-3 has been identified as another important inhibitory receptor expressed by exhausted CD8+ T cells. In mouse models of cancer, it has been shown that the most dysfunctional tumour-infiltrating CD8+ T cells actually co-express PD-1 and TIM-3.

LAG-3 is another recently identified inhibitory receptor that acts to limit effector T-cell function and augment the suppressive activity of T regulatory cells. It has recently been revealed that PD-1 and LAG-3 are extensively co-expressed by tumour-infiltrating T cells in mice, and that combined blockade of PD-1 and LAG-3 provokes potent synergistic antitumor immune responses in mouse models of cancer.

PD-1 pathway blockade can be combined with vaccines or other immunomodulatory antibodies for improved therapeutic efficacy. Currently, antagonist mAbs against both PD-1 and their ligand PD-L1 are in various stages of development for the treatment of cancer, and recent human trials have shown promising results in cancer patients with advanced, treatment refractory disease.

The first of the agents blocking the B7-H1/PD-1 pathway to enter phase I clinical trials was Nivolumab (MDX-1 106/BMS-936558/ONO-4538), a fully human IgG4 anti-PD-1 mAb developed by Bristol-Myers Squibb. Another PD-1 mAb undergoing clinical evaluation is CT-01 1, a humanized IgG1 mAb specific for PD-1 developed by CureTech Ltd. Other agents include Lambrolizumab (MK-3475-Merck), a humanized monoclonal IgG4 PD-1 antibody; BMS-936559, a fully human IgG4 PD-L1 antibody and Roche's MPDL3280A, a human monoclonal antibody that targets the PD-L1 pathway. The results of a phase II study that compared combined nivolumab and ipilimumab with ipilimumab alone in patients with F wild-type melanoma showed objective response rates of 61% with the combination therapy and 11% with the monotherapy, with complete responses in 22% and 0% of patients, respectively. Treatment-related adverse events of grade 3 or 4 were reported in 54% of the patients in the combination group and in 24% of those in the ipilimumab group. Treatment-related adverse events of any grade that led to discontinuation of the study drug occurred in 7.7% of the patients in the nivolumab group, 36.4% of those in the nivolumab-plus-ipilimumab group, and 14.8% of those in the ipilimumab group.

Anti-cancer immune responses accompanying the action of chemo- and radiotherapy have been recently reviewed and show that such responses are critical to therapeutic success by eliminating residual cancer cells and maintaining micrometastases in a state of dormancy (Zitvogel et al, J Clin Invest 2008; 118: 1991-2001). However, this reference makes it clear that there is no simple immunotherapeutic strategy available for consistently enhancing such immune responses.

There is evidence that therapeutic procedures that induce certain forms of immunogenic cancer cell death also lead to release of tumour antigens. There are three main types of cell death (Tesniere et al, Cell Death Differ 2008; 15:3-12): apoptosis (type 1), autophagy (type 2) and necrosis (type 3). Apoptosis, or programmed cell death, is a common and regular occurring phenomenon essential for tissue remodelling, especially in utero but also ex utero. It is characterized by DNA fragmentation in the nucleus and condensation of the cytoplasm to form ‘apoptotic bodies’ which are engulfed and digested by phagocytic cells. In autophagy, cell organelles and cytoplasm are sequestered in vacuoles which are extruded from the cell. Although this provides a means of survival for cells in adverse nutritional conditions or other stressful situations, excess autophagy results in cell death. Necrosis is a ‘cruder’ process characterized by damage to intracellular organelles and cell swelling, resulting in rupture of the cell membrane and release of intracellular material.

Necrosis has been principally classified as immunogenic cell death. A limited number of studies indicate that procedures inducing immunogenic cell death release mediators and tumour antigens that are able to both induce immune responses, including activation of cytotoxic CD8+ T cells and NK cells and act as targets, including rendering antigens accessible to Dendritic Cells (DC), able in principle to create an in vivo DC vaccine.

It is more useful to categorize cell death into immunogenic and non-immunogenic forms, irrespective of the precise mechanism of such cell death. In a therapeutic setting with restoration of beneficial immune regulation, antigens released by immunogenic cell death would then be able to elicit effective anti-tumour immune responses, particularly if they are release in the presence of Danger-Associated (or Damage-Associated) Molecular Pattern (DAMP) (Jerome et al., N. Eng. J. Med. 2004; 350: 41141-2).

Radiotherapy is required by 60-70% of cancer patients during their treatment. Radiotherapy is the most widely used and effective anti-tumour therapy, but it can cause acute and late damage to healthy tissues. The dose delivered to the tumor is, hence, limited by the toxicity to nearby healthy tissue; this can mean that a tumor cannot be completely killed, and the efficacy of radiotherapy will be decreased. Thus, preventing or mitigating radiation-induced healthy tissue injury has always been a topic of particular interest in radiotherapy research. Modern radiotherapy technologies, such as intensity-modulated radiation therapy (IMRT), helical tomotherapy (HT), and proton radiotherapy can reduce radiation damage to healthy tissues. Furthermore, as the number of long-term cancer survivors increases, late onset toxicities resulting from RT are emerging that significantly impact the quality of life of those patients. Consequently, there is a need for novel RT strategies that maintain the anti-tumor effect whilst limiting the extent of toxicities induced in the surrounding healthy tissue. Limiting the induction of toxicities to normal tissue would subsequently increase the therapeutic index of RT regimes. radiotherapy uses an external radiation beam where the dose decreases exponentially but which can deposit energy within a certain depth of the patient tissue. Therefore, in the cases of deep-seated tumours, the healthy normal tissue in front of the tumour receives a large dose of ionizing radiation relative to the tumour. Furthermore, it is possible that healthy normal tissue located behind the tumour can receive an exit dose of radiation if the beam passes through the tumour. This can present significant challenges to sensitive tissues and organs at risk (OAR), such as the brain and spinal cord.

Advancements in modern radiotherapy deliverance and imaging techniques such as image-guided radiotherapy, intensity-modulated radiotherapy, and volumetric modulated arc therapy, along with targeted combinatorial drug therapies and immunotherapy, have increased the therapeutic index of radiotherapy. Furthermore, the increased use of proton beam therapy (PBT) which displays a lower entrance dose compared to conventional radiotherapy and where the majority of the radiation dose can be specifically targeted at the tumor, can also limit the unnecessary irradiation of surrounding normal tissues leading to reduced adverse side-effects. Despite this, many tumours remain intrinsically radioresistant and therefore further discovery and research into novel treatment strategies is critical to maximize the tumour-killing effect of radiotherapy, while simultaneously minimizing the toxic impact to surrounding normal tissues. Stereotactic Radiosurgery has been used for several decades to treat various neurological disorders such as brain metastasis, solitary primary brain tumors, arteriovenous malformations (AVM), pituitary adenoma and acoustic neuroma. It has more recently been used to treat some functional disorders such as Parkinson's disease.

Current Stereotactic Radiosurgery devices consist of customized linear accelerators, such as devices provided by BrainLab AG and Accuray Incorporated, or devices using 201 Cobalt sources, such as devices provided Elekta Ltd. These devices are used to deliver very high radiation dose to ablate the tumor/lesion in a single treatment, or a small number of daily treatments.

Intended lesions are often situated in close proximity to the other functionally sensitive areas of the cranium. Thus, a very high geometric accuracy in aligning the target volume encompassing the lesion(s) is needed to ensure that the radiation is delivered substantially to the lesion(s) and negligibly everywhere else.

The objective of the radiation therapy is to target the lesion with a high dose of radiation with minimal impact on all the surrounding normal tissue. An initial treatment planning procedure is performed prior to external beam RT delivery to localize the tumour and other critical structures surrounding the tumour. This planning procedure typically involves CT imaging to identify these structures. Based on the segmented tumour and surrounding tissue structures, a set of beam orientations and collimator settings are developed through an iterative process to determine the optimal dose distribution pattern that maximizes dose to the tumour whilst minimizing dose to surrounding critical avoidance structures.

MRI is currently the optimal modality for tumour localization based on the higher soft-tissue contrast, compared with CT, and can be incorporated into the treatment planning workflow. Although MRI provides good location of the tumour for treatment planning purposes, these treatment planning images are normally collected several days prior to treatment, and, as such, may not be completely representative of tumour location on the day of treatment. To address this limitation, oncologists tend to increase the target volume ensure all the tumor tissue receives the maximum dose. The expectation is that all cells in the targeted region will receive the required RT treatment dose, and that this increased treatment target volume will lessen the impact of errors between treatment planning dose distribution, and the dose delivered to the actual region of the lesion. However, this increased treatment margin also produces collateral tissue damage that may have a significant impact of the quality of life of the patient and increase the possibility of secondary RT-induced cancer.

To mitigate the need for increased treatment margins, clinicians have employed a method referred to as image-guided external beam radiation therapy, in which an image is acquired immediately prior to RT treatment delivery. One such available solution involves completely integrating the MRI system with a linear accelerator to enable real-time imaging of the tumour during RT treatment. However, this design is complex, expensive and may involve serious compromises on the functional performance of both the MRI and linear accelerator.

The treatment planning images are typically collected days prior to the actual fractionated treatment delivery that can occur over the course of several weeks. As such, the position of the tumour in the treatment imaging plans may not be representative of the actual lesion position on each day of treatment. By incorporating image guidance immediately before each treatment session, it is possible to determine the exact position of the lesion within each treatment session. Acquiring MR images immediately before RT treatment would identify the exact lesion location, and define the correct gantry positions for conformal radiation delivery. A radiotherapy device generally includes a linear electron beam accelerator which is mounted on a gantry and which can rotate about an axis which is approximately parallel to the patient lying on the patient couch. The patient is treated using either an electron, γ- or X-Ray beam produced from the original electron beam. The beam is focused at a target by the combination of the use of a collimator and the rotation of the beam. The patient is placed on a couch which can be positioned such that the target lesion can be located in the plane of the electron beam as the gantry rotates. This patient couch is designed to adjust the position of the patient to align the targeting exactly at the isocentre of the RT system using up to six degrees of motion (x, y, z, roll, pitch, and yaw). The current couch designs employed by several manufacturers employ a cantilevered couchtop that enables a sufficient range of motion to treat disease sites throughout the entire body.

Cancer patients are often assessed against standard criteria for acknowledging how much a disease impacts a patient's daily living abilities. This assessment is known as the patient's Performance Status (PS). The most popular standardised assessment was developed by the Eastern Cooperative Oncology Group (ECOG) and published in 1982. The ECOG Scale describes a patient's level of functioning, or Performance Status, in terms of their ability to care for themselves, daily activities and physical abilities such as walking or working. It is also a way for medical professionals to track changes in a patient's level of functioning as a result of interventions during cancer treatment. A score of PS0 indicates the absence of disease, and a score of PS5 indicates death.

IMM-101 is a heat killed Mycobacterium obuense, typically prepared as a suspension in borate buffered saline which modulates innate and adaptive immune systems, restores Type 1 immune responses and may downregulate Type 2 immune responses. In murine genetically engineered mouse models of pancreatic cancer, IMM-101 prolonged survival. IMM-101 also induced an activation of T cells and cytotoxic CD8+ cells at tumour sites, in the periphery and systemically. It has been demonstrated that the survival benefit was derived from CD8+ T cells from a depletion experiment using a neutralizing antibody. IMM-101 contains microbial-associated molecular patterns (MAMPs) that activate a defined selection of pathogen recognition receptors (PRRs) including toll like receptor (TLR) ½ on innate immune cells like dendritic cells (DCs) (Bazzi et al. 2017, Galdon et al. 2019). IMM-101 activation of immature DCs leads to the skewed maturation of activated cDC1, of which activation predominantly induces a type 1 immune response defined by the generation and maturation of IFN-γ, perforin and granzyme producing CTLs (Galdon et al., 2019), required for effective tumour cell killing. IMM-101 induced cDC1 activation also results in the generation of activated IFN-γ producing Th-1 cells NK, NKT and γδ-T cells (Fowler et al., 2014; Galdon et al., 2019), which can kill tumor cells by different mechanisms. Activated γδ-T cells are efficient antigen presenting cells (Moser et al., 2017), which may further boost anti-tumor responses. IMM-101 also activate other innate immune cells, including monocytes, which mature into M1 macrophages (Bazzi et al. 2015) that can enhance anti-tumour responses and prevent the formation of immune-suppressive M2 macrophages.

When using a pancreatic cancer mouse model (mutations in Kras, p53 and Pdx-Cre, known as KPC), the combination of gemcitabine and IMM-101 did not prolong survival, however this combination significantly reduced formation of metastases in the liver and peritoneum. In a phase II trial involving metastatic pancreatic cancer patients, overall survival was significantly improved from 4.4 to 7.0 months with the addition of IMM-101 to gemcitabine therapy and some long-term survivors were seen. In addition, IMM-101 was able to stimulate and mature dendritic cells into activated phenotype through a Type 1 biased stimulation. This Type-1 immune response has also been shown to be a prerequisite for optimal efficacy of immune checkpoint inhibitors (CPI) as the CD103(+) dendritic cells were the only antigen presenting cells transporting intact antigens to the lymph nodes and priming tumour-specific CD8(+) T cells. Using an in vivo bone marrow derived cells (BMDC) transfer model, it has been demonstrated that administration of IMM-101 treated dendritic cells (DCs) induces IFN gamma and IL-17 and enhances DC antigen processing and presentation ability. It was also demonstrated that administration of IMM-101 in combination with PD-1 antibodies reduces tumour burden in syngeneic models of breast and pancreatic cancer, through the significant increase of intratumoral CD8 T cell/Treg ratio in combination with anti PD-1 compared to anti PD-1 alone.

The function of APCs in antitumor immunity is to transfer tumour antigens to tumour-draining lymph nodes for tumor-specific CD8+ T-cell priming. In melanoma, CD103+ DCs are the only APCs that have such a function. In melanoma mouse models, administration of the growth factor FLT3L and poly I:C has expanded and activated CD103+ DC progenitors in the tumour, thereby reversing anti-PD-L1 resistance. Furthermore, studies show that failed accumulation of CD103+ dendritic cells, a cell type that is the major source of the T cell-recruiting chemokines CXCL9/10, in non-inflamed tumours mediates deficient entry of therapeutically activated T cells and immunotherapy resistance. Therefore, absence of CD103+ DCs from the tumour microenvironment may be a dominant mechanism of resistance to multiple immunotherapies.

Conventional dendritic cells (cDCs) are specialized antigen-presenting cells that control T cell immunity. Lineage tracing experiments in mice have mapped two developmentally and functionally distinct populations, cDC1 and cDC2, that reside in peripheral tissues where they are defined by expression of CD103 and CD11b, respectively. These lineages and their functions are conserved in humans. Of these, cDC1 are highly efficient at cross-presenting antigens to cytotoxic T cells and are the major stimulatory cDC population within tumours, both for the generation of anti-tumor immunity in draining lymph nodes (LNs) and upon direct interaction with effector T cells within the tumor microenvironment. Furthermore, cDC1 are critical for therapeutic responses to checkpoint blockade.

Although these combinations have demonstrated efficacy in the treatment of various cancers, including pancreatic cancer, there are still a lack of defined treatment regimens which are appropriate for those patients who are deemed too vulnerable or unfit to receive efficacious chemotherapy due to their toxicity profiles.

In pancreatic cancer, FOLFIRINOX (a mixture of folinic acid, fluorouracil, irinotecan hydrochloride and oxaliplatin), nab-paclitaxel (Abraxane®) plus gemcitabine are the preferred first line treatments for metastatic pancreatic ductal adenocarcinoma (PDAC) patients with an ECOG Performance Status of 0 and 1 only. These treatments are contraindicated for patients with a score of PS2 or above, due to their toxicity profile. However, almost 50% of patients presenting with de novo metastatic disease are of a lower performance score and have very limited options in terms of active intervention. Single agent chemotherapeutics such as gemcitabine confer a modest benefit, at best, in the trade-off between an already limited quality of life and coping with a toxic regimen that may worsen the patient's health. This subgroup of patients are traditionally poorly served in the clinical trial setting due to these inclusion and exclusion criteria. Therefore, a novel tolerable combination therapy is urgently needed for at least such PS2, 3 and 4 patients but also beneficial for PS 0 and PS1 patients, too.

### SUMMARY OF THE INVENTION

The present invention provides an effective method of treating, reducing, inhibiting or controlling cancer in a subject, by administering a Mycobacterium preparation to a patient in need thereof, in combination with one or more doses of ionizing radiation selected from photon therapy or particle therapy.

In a first aspect of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium.

In a second aspect of the invention, there is provided a method of treating or controlling cancer comprising a primary tumour in a patient, wherein said method comprises simultaneously, separately or sequentially administering to the subject, (i) one or more doses of ionizing radiation selected from photon therapy or particle therapy to the patient, and (ii) a non-pathogenic non-viable Mycobacterium, wherein said method results in enhanced therapeutic efficacy relative to administration of one or more doses of ionizing radiation selected from photon therapy or particle therapy, or non-pathogenic non-viable whole cell Mycobacterium alone.

The present invention therefore provides a non-pathogenic non-viable Mycobacterium for use in the treatment, reduction, inhibition or control of cancer in a subject, together with a method of treating said cancer by administration of said Mycobacterium. The inventors have unexpectedly found that the administration of Mycobacterium to a patient in combination with ionizing radiation, results in greater efficacy compared to either agent alone.

### DETAILED DESCRIPTION OF THE INVENTION

The invention provides an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium.

It is based upon the surprising discovery that administration of a non-pathogenic whole cell heat-killed Mycobacterium as a neoadjuvant treatment prior to surgery or checkpoint inhibitor therapy (CPI), in combination with targeted ionizing radiation/radiotherapy (RT), results in enhanced anti-tumour activity and/or antitumor activity that is more potent than undertaking the surgery, RT or CPI alone. Further, treatment with the Mycobacterium was shown to result in an effective and sustained pathological response and survival of treated patients.

In a phase II clinical trial involving patients with pancreatic ductal adenocarcinoma (PDAC), it was found that administering non-pathogenic whole cell heat-killed Mycobacterium in a multimodality neoadjuvant treatment regimen combined with the chemotherapy agent gemcitabine, resulted in a surprisingly effective and sustained pathological response and survival relative to gemcitabine alone, particularly in metastatic patients.

Accordingly, whilst some neoadjuvant therapies are known in the art to aid resection surgeries or other cancer treatments, the invention disclosed herein provides specific immunomodulator-based neo, peri- and adjuvant protocols which are optimised to improve therapeutic efficacy and thus responses in a greater proportion of subjects that undergo surgery and/or checkpoint immunotherapy.

In order that the present invention may be more readily understood, certain terms are first defined. Additional definitions are set forth throughout the detailed description.

The term “neoadjuvant” or “neoadjuvant therapy”, which may be used interchangeably with “preoperative therapy”, refers to the administration of one or more therapeutic agents or modalities prior to a main treatment or surgery. It is the aim of such a therapy to effectively reduce the difficulty and morbidity of more extensive surgical procedures, as well as enhance the overall efficacy of said surgical procedures.

The terms “adjuvant therapy” and “peri-adjuvant therapy” are also used herein, which refer to therapies following surgery or treatment, or, both prior to and following surgery or treatment, respectively.

A “tumour resection surgery”, which may be referred to herein simply as “surgery” or “surgical procedure”, refers to procedures that aim to physically remove all or part of a tumour, preferably a primary tumour or node, or isolated, single tumour or node. Tumour resection surgery is often used before chemotherapy or radiation, and often in a regimen involving adjuvant therapy.

The term tumour resection surgery is used to define a variety of forms of tumour resection surgeries, and may further include ancillary surgeries, including those of lymph node resection nature, such as lymphadenectomy. It may also encompass surgery on a localised metastatic tumour.

The terms “tumour,” “cancer” and “neoplasia” may be used interchangeably and refer to a cell or population of cells whose growth, proliferation or survival is greater than growth, proliferation or survival of a normal counterpart cell, e.g. a cell proliferative or differentiative disorder. Typically, the growth is uncontrolled. The term “malignancy” refers to invasion of nearby tissue. The term “metastasis” refers to spread or dissemination of a tumour, cancer or neoplasia to other sites, locations or regions within the subject, in which the sites, locations or regions are distinct and/or distant from the primary tumour, node or cancer.

The term “non-metastatic” refers to where, relative to a primary tumour, node or cancer in a patient, there are no distant metastases or residual disease, as determined by CT, MRI or Positron emission tomography (PET) with 2-deoxy-2-[fluorine-18] fluoro-D-glucose (18F-FDG) scanning.

A “checkpoint inhibitor” is an agent which acts on surface proteins which are members of either the TNF receptor or B7 superfamilies, including agents which bind to negative co-stimulatory molecules, selected from a cell, protein, peptide, antibody ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1 R, CD94/NKG2A, TDO, TNFR, DcR3, CD27, CD28, CD40, CD122, CD137, OX40, GITR, ICOS and combinations thereof. A “blocking agent” is an agent which either binds to the above costimulatory molecules and/or their respective ligands. “Checkpoint inhibitor” and “blocking agent” can be used interchangeably throughout. The inhibitor is preferably an antibody or antigenic-binding molecule that targets an antigenic site on the surface proteins. For example, the inhibitor is an antibody that targets an antigenic site on PD-L1, or PD-1 or CTLA-4.

The terms “Programmed Death 1,” “Programmed Cell Death 1,” “Protein PD-1,” “PD-1,” and “PD1,” are used interchangeably, and include variants, isoforms, species homologs of human PD-1, and analogs having at least one common epitope with PD-1. The complete PD-1 sequence can be found under GenBank Accession No. U64863.

The terms “OX40”, “CD137” and “OX-40” are used interchangeably, and include variants, isoforms, species homologs of human OX40, and analogs having at least one common epitope with OX40.

The terms “GITR” and “Glucocorticoid-Induced TNFR family Related Gene” are used interchangeably, and include variants, isoforms, species homologs of human GITR, and analogs having at least one common epitope with GITR.

The terms “CD137” and “4-1 BB” are used interchangeably, and include variants, isoforms, species homologs of human CD137, and analogs having at least one common epitope with CD137.

The terms “B7-H3 and “CD276” are used interchangeably, and include variants, isoforms, species homologs of human B7-H3, and analogs having at least one common epitope with B7-H3.

The terms “B7-H4” and “VTCN1” are used interchangeably, and include variants, isoforms, species homologs of human B7-H4, and analogs having at least one common epitope with B7-H4.

The terms “A2AR” and “Adenosine A2A receptor” are used interchangeably, and include variants, isoforms, species homologs of human A2AR, and analogs having at least one common epitope with A2AR.

The terms “IDO” and “Indoleamine 2,3-dioxygenase” are used interchangeably, and include variants, isoforms, species homologs of human IDO, and analogs having at least one common epitope with IDO.

The terms “cytotoxic T lymphocyte-associated antigen-4,” “CTLA-4,” “CTLA4,” and “CTLA-4 antigen” are used interchangeably, and include variants, isoforms, species homologs of human CTLA-4, and analogs having at least one common epitope with CTLA-4.

As used herein, “sub-therapeutic dose” means a dose of a therapeutic compound (e.g., an antibody) or modality or duration of therapy which is lower than the usual or typical dose of the therapeutic compound or therapy or modality of shorter duration, when administered alone for the treatment of cancer.

The term “therapeutically effective amount” is defined as an amount of one or more therapeutic agents or modalities, including checkpoint inhibitors, in combination with an immunomodulator, that preferably results in a decrease in severity of disease symptoms, an increase in frequency and duration of disease symptom-free periods, or a prevention of impairment or disability due to the disease affliction. The terms “effective amount” or “pharmaceutically effective amount” refer to a sufficient amount of an agent to provide the desired biological or therapeutic result. That result can be reduction, amelioration, palliation, lessening, delaying, and/or alleviation of one or more of the signs, symptoms, or causes of a disease, or any other desired alteration of a biological system. In reference to cancer, an effective amount may comprise an amount sufficient to cause a tumour to shrink and/or to decrease and/or stabilise the growth rate of the tumour (such as to suppress tumour growth) or to prevent or delay other unwanted cell proliferation. In some embodiments, an effective amount is an amount sufficient to delay development, or prolong survival or induce stabilisation of the cancer or tumour.

In some embodiments, a therapeutically-effective amount is an amount sufficient to prevent or delay recurrence. A therapeutically-effective amount can be administered in one or more administrations. The therapeutically-effective amount of one or more therapeutic agents or modalities or combinations thereof, may result in one or more of the following: (i) reduce the number of cancer cells; (ii) reduce tumour size; (iii) inhibit, retard, slow to some extent and preferably stop cancer cell infiltration into peripheral organs; (iv) inhibit (i.e., slow to some extent and preferably stop) tumour metastasis; (v) inhibit tumour growth; (vi) prevent or delay occurrence and/or recurrence of tumour; and/or (vii) relieve to some extent one or more of the symptoms associated with the cancer. For example, for the treatment of tumours, a “therapeutically effective dosage” may induce tumour shrinkage by at least about 5% relative to baseline measurement, such as at least about 10%, or about 20%, or about 60% or more. The baseline measurement may be derived from untreated subjects.

A therapeutically-effective amount of a therapeutic compound can decrease tumour size, or otherwise ameliorate symptoms in a subject. One of ordinary skill in the art would be able to determine such amounts based on such factors as the subject's size, the severity of the subject's symptoms, and the particular composition or route of administration selected. The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells.

The terms “effective amount” or “pharmaceutically effective amount” refer to a sufficient amount of an agent to provide the desired biological or therapeutic result. That result can be reduction, amelioration, palliation, lessening, delaying, and/or alleviation of one or more of the signs, symptoms, or causes of a cancer or any other desired alteration of a biological system. In reference to cancer, an effective amount may comprise an amount sufficient to cause a tumour to shrink and/or to decrease the growth rate of the tumour (such as to suppress tumour growth) or to prevent or delay other unwanted cell proliferation. In some embodiments, an effective amount is an amount sufficient to delay development, or prolong survival or induce stabilisation of the cancer or tumour. Preferably, therapeutic efficacy is measured by a decrease or stabilisation of tumour size of one or more said tumours, as defined by RECIST 1.1, including stable diseases (SD), a complete response (CR) or partial response (PR) of the target tumour; and/or stable disease (SD) or complete response (CR) of one or more non-target tumours. Alternatively, therapeutic efficacy is assessed by Immune Related Response Criteria (irRC), iRECIST or irRECIST, as would be known to the skilled person.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells.

The term “checkpoint inhibitor” or “immunomodulator” or “immunotherapy” may further include use of a cell, virus, lysate, vector, gene, mRNA, DNA, nucleic acid, protein, polypeptide, peptide, antibody, bispecific antibody, multi-specific antibody, ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, in practising the invention.

The term “antibody” as referred to herein includes whole antibodies and any antigen-binding fragment (i.e., “antigen-binding portion”) or single chains thereof. The term “antigen-binding portion” of an antibody (or simply “antibody portion”), as used herein, refers to one or more fragments of an antibody that retain the ability to specifically bind to a receptor and its ligand (e.g., PD-1). including: (i) a Fab fragment, (ii) a F(ab′) 2 fragment; (iii) a Fd fragment consisting of the VH and CHI domains; (iv) a Fv fragment, (v) a dAb fragment (Ward et al, Nature, 341:544-546 (1989)), which consists of a VH domain; and (vi) an isolated complementarity determining region (CDR). Single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences.

The term “humanized antibody” is intended to refer to antibodies in which CDR sequences derived from the germline of another mammalian species, such as a mouse, have been grafted onto human framework sequences. Additional framework region modifications may be made within the human framework sequences. In addition to antibodies, other biological molecules may act as checkpoint inhibitors, including peptides having binding affinity to the appropriate target.

By “non-viable”, it is meant that the Mycobacterium have been microbiologically inactivated through certain means of cell-killing. Methods to enable or enforce such non-viability may include heat-killing, extended freeze-drying (Tolerys SA), irradiation by gamma waves or electron beam, or subjecting the mycobacteria to chemicals such as formaldehyde. Such preparation during manufacture would mean the organism is not associated with side-effects known from delivering live or attenuated organisms.

The term “treatment” or “therapy” refers to administering an active agent with the purpose to cure, heal, alleviate, relieve, alter, remedy, ameliorate, improve, or affect a condition (e.g., a disease), the symptoms of the condition, or to prevent or delay the onset of the symptoms, complications, biochemical indicia of a disease, or otherwise arrest or inhibit further development of the disease, condition, or disorder in a statistically significant manner.

As used herein, the term “subject” or “patient” is intended to include human and non-human animals. Preferred subjects include human patients in need of enhancement of an immune response. The methods are particularly suitable for treating human patients having a disorder that can be treated by augmenting the T-cell mediated immune response. In a particular embodiment, the methods are particularly suitable for treatment of cancer cells in vivo.

As used herein, the terms “concurrent administration” or “concurrently” or “simultaneous” mean that administration occurs on the same day. The terms “sequential administration” or “sequentially” or “separate” mean that administration occurs on different days.

“Simultaneous” administration, as defined herein, includes the administration of the immunomodulator and one or more therapeutic agents or modalities, including checkpoint inhibitor therapy, within about 2 hours or about 1 hour or less of each other, even more preferably at the same time.

“Separate” administration, as defined herein, includes the administration of the immunomodulator and one or more therapeutic agents or modalities, including checkpoint inhibitor therapy, more than about 12 hours, or about 8 hours, or about 6 hours or about 4 hours or about 2 hours apart.

“Sequential” administration, as defined herein, includes the administration of the immunomodulator and one or more therapeutic agents or modalities, including checkpoint inhibitor therapy or chemotherapeutic agents, each in multiple aliquots and/or doses and/or on separate occasions. The immunomodulator may be administered to the patient after before and/or after administration of the one or more therapeutic agents or modalities, including checkpoint inhibitor therapy. Alternatively, the immunomodulator is continued to be applied to the patient after treatment with one or more therapeutic agents or modalities, including checkpoint inhibitor therapy.

The use of the alternative (e.g., “or”) should be understood to mean either one, both, or any combination thereof of the alternatives. As used herein, the indefinite articles “a” or “an” should be understood to refer to “one or more” of any recited or enumerated component.

As used herein, “about” means within an acceptable error range for the particular value as determined by one of ordinary skill in the art, which will depend in part on how the value is measured or determined, i.e., the limitations of the measurement system. For example, “about” can mean within 1 or more than 1 standard deviation per the practice in the art. Alternatively, “about” can mean a range of up to 20%. When particular values are provided in the application and claims, unless otherwise stated, the meaning of “about” should be assumed to be within an acceptable error range for that particular value.

As part of the invention, the subject is classed as having a performance status of 0, 1, 1-, 2, 3 or 4 according to the ECOG scale. The ECOG scale may also be known as the Zubrod Scale or the WHO Scale, and is therefore interchangeable with such terms. The ECOG scale is from PS0 to PS5, as detailed below. In some embodiments, a value of PS1—represents an integer between PS1 and PS2, whereby the patient is not exhibiting all aspects of behaviour as severe as PS2, and is not exhibiting all aspects of behaviour categorised as PS1. A further clinical classification method of performance status is also known as the Karnofsky Scale, which is from 0 to 100, whereby 100 is the absence of disease and 0 is death.

Two clinical observers are usually required to assess performance status. If there is any discrepancy between the two scores, the highest (worst) assessment will be used.

| | Karnofsky | ECOG | |
|---|---|---|---|
| Karnofsky Status | Grade | Grade | ECOG Status |
| Normal, no complaints | 100 | 0 | Fully active, able to carry on all |
| | | | pre-disease performance without |
| | | | restriction |
| Able to carry on normal | 90 | 0 | Restricted in physically |
| activities. Minor signs or | | | strenuous activity but ambulatory |
| symptoms of disease | | | and able to carry out work of a |
| | | | light or sedentary nature, e.g., |
| | | | light housework, office work |
| Normal activity with effort | 80 | 1 | Restricted in physically |
| | | | strenuous activity but ambulatory |
| | | | and able to carry out work of a |
| | | | light or sedentary nature, e.g., |
| | | | light housework, office work |
| Care for self. Unable to | 70 | 1 | Ambulatory and capable of all |
| carry on normal activity or | | | selfcare but unable to carry out |
| to do active work | | | any work activities. Up and about |
| | | | more than 50% of waking hours |
| Requires occasional | 60 | 2 | Ambulatory and capable of all |
| assistance, but able to | | | selfcare but unable to carry out |
| care for most of his needs | | | any work activities. Up and about |
| | | | more than 50% of waking hours |
| Requires considerable | 50 | 2 | Capable of only limited selfcare, |
| assistance and frequent | | | confined to bed or chair more |
| medical care | | | than 50% of waking hours |
| Disabled. Requires | 40 | 3 | Capable of only limited selfcare, |
| special care and | | | confined to bed or chair more |
| assistance | | | than 50% of waking hours |
| Severely disabled. | 30 | 3 | Completely disabled. Cannot |
| Hospitalisation indicated | | | carry on any selfcare. Totally |
| though death non- | | | confined to bed or chair |
| imminent | | | |
| Very sick. Hospitalisation | 20 | 4 | Completely disabled. Cannot |
| necessary. Active | | | carry on any selfcare. Totally |
| supportive treatment | | | confined to bed or chair |
| necessary | | | |
| Moribund | 10 | 4 | Completely disabled. Cannot |
| | | | carry on any selfcare. Totally |
| | | | confined to bed or chair |
| Dead | 0 | 5 | Dead |

A Karnofsky Scale value of 60 or above may be equivalent to an ECOG Scale reading of PS2, 3, 4 or 5. Alternatively, the subject or patient targeted for administration with the Mycobacterium can be alternatively classified as not being sufficiently fit to tolerate two or more chemotherapy regimens. A subject classified as not being sufficiently fit to tolerate two or more chemotherapy regimens is considered to be equivalent to a subject or patient with a score of PS 2, 3, 4 or 5, according to the ECOG scale, or a subject or patient with a score of 60 or above according to the Karnofsky Scale, e.g. 70.

A chemotherapy regimen according to the present invention is an administration of a chemotherapeutic agent or multiple chemotherapeutic agents, optionally simultaneously, separately or sequentially with any other therapeutic agent. The regimen may be repeated more than once a day, more than once a week, more than once a month, or more than once a year. The chemotherapeutic agent may be cytotoxic, which is defined as likely to cause cellular death by interfering in one or more cellular mechanisms.

M. vaccae and M. obuense have been shown to induce a complex immune response in the host. Treatment with these preparations will stimulate innate and type-1 immunity, including Th1 and macrophage activation and cytotoxic cell activity. They also independently down-regulate inappropriate Th2 responses via immunoregulatory mechanisms. It has been shown in experiments with mouse and human immune cells that IMM-101 (a non-viable, whole cell M. obuense) is a strong activator of antigen presenting macrophages and dendritic cells (DCs) and that the DC activation leads to a typical Type 1 immune response, with formation and activation of CD4+ T-helper 1 lymphocytes (Th1s) and CD8+ cytotoxic T lymphocytes (CTLs) and increased production of the cytokine interferon-γ (IFN-γ) in the lymph nodes in which IMM-101 activated DCs are present.

In addition, other experiments have shown that IMM-101 also increases the number and activation of natural killer cells (NKs) and T cells expressing gamma/delta receptors (γδ-T cells). Th1s and CTLs require tumour cells to express specific tumour-associated antigens (TAAs) for their attack, whereas NK and γδ-T cells do not require the presence of such TAAs to kill tumour cells. These four different immune cells work in concert to form an effective anti-tumour response. In relation to cancer, it is likely that the formation of IFN-γ producing CTLs is the most important result from IMM-101 treatment, since the observed anti-tumour effect of IMM-101 could be completely abrogated by the depletion of CD8+ T cells in a pancreas cancer model.

IMM-101's ability to activate macrophages may not only assist in the activation of DCs through the release of pro-inflammatory macrophage-derived cytokines (such as IL-12 required for skewing DCs into Type 1 immune responses), but may also be of importance for changing tumour associated immunosuppressive type 2 macrophages into tumour aggressive type 1 macrophages. This latter feature was shown for a similar heat-killed mycobacterium, M. indicus pranii.

An important feature of IMM-101 is its ability to activate and mature DCs into a sub-class of dendritic cells known as cDC1s (i.e. DCs that are required for Type 1 immune responses). It has been shown that activation of sufficient numbers of cDC1s is a prerequisite for CPIs to be effective.

It is generally believed that a Type 1 immune response resulting in INF-γ producing Th1s and CTLs, which specifically attack TAAs-expressing tumour cells, and activated NK and γδ-T cells, which attack tumours through other mechanisms, is the body's main mechanism and a pre-requisite for an effective anti-cancer response and should therefore be at the core of any immune-mediated cancer treatment—preclinical data show that IMM-101 is capable of stimulating such required Type 1 immune responses.

The impact of IMM-101 on DC priming has been studied in vitro and it was found that IMM-101 displayed a dose-dependent ability to induce phenotypic activation and cytokine production for both human and murine DCs. For example, GM-CSF derived murine DC displayed a dose dependent response to IMM-101, with elevated membrane expression of CD80, CD86, CD40 and MHC II and increased production of IL-6, IL-12p40 and nitric oxide, which are all molecules that are essential for effective antigen-dependent activation of T cells. Moreover, human monocyte-derived DCs showed a similar response to IMM-101, with up-regulation of CD80, CD86 and MHC II and secretion of a number of relevant cytokines, showing clear activation of DCs. Exposure to IMM-101 in vitro also showed that IMM-101 functionally affects the DCs by enhancing their ability to process and present antigen.

In vivo experiments have shown that IMM-101 activated DCs are able to activate CD8+ and CD4+ T cells and promote secretion of IFN-γ following re-stimulation of draining lymph node cell preparations, 7 days after subcutaneous adoptive transfer of IMM-101 (in vitro) activated GM-CSF derived murine DCs into naïve recipient mice.

Previous clinical studies have demonstrated the safety of IMM-101 as monotherapy and in combination with chemotherapy agents or checkpoint inhibitors (Stebbing et al. 2012, Dalgleish et al. 2016, Dalgleish et al. 2018).

Preoperative short time administration of checkpoint inhibitors in microsatellite instability (MSI) high colorectal cancers has been shown to induce high rates of pathological regression. In a recently presented small explorative phase II study (Chalabi et al. 2018, immunotherapy of cancer 29:8), six weeks of preoperative administration of the CTLA-4 antibody ipilimumab and the PD-1 antibody nivolumab was shown to result in a complete or subtotal pathological remission, suggesting that neoadjuvant therapies could be particularly efficacious for some cancer presentations. The present invention thus provides a protocol to use IMM-101 as a surprisingly favourable neoadjuvant treatment, or adjuvant treatment, with or without the use of other chemotherapy agents, to improve the unfavourable prognosis of patients intended to undergo tumour resection surgery or checkpoint inhibitor therapy for cancer. Such patients may include those with colorectal cancer, particularly mismatch repair-deficient (dMMR) colorectal cancers with microsatellite instability (MSI).

In a further aspect of the invention, the cancer determined as being microsatellite instability (MSI) high, as measured by a PCR-based assay and/or IHC staining for DNA mismatch repair (MMR) protein expression.

In a preferred embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, and wherein said photon therapy comprises administration of x-rays and/or gamma rays.

In a further preferred embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, and wherein said particle therapy comprises administration of electrons, protons, neutrons, pions, neon ions, argon ions, silicon ions or carbon ions.

In another embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, and wherein said photon therapy comprises administration of x-rays and/or gamma rays, wherein said particle therapy comprises administration of electrons, protons, neutrons, pions, neon ions, argon ions, silicon ions or carbon ions, and wherein said one or more doses of ionizing radiation selected from photon therapy or particle therapy is to be administered to the patient from either an external or internal source.

In an embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, and wherein said photon therapy comprises administration of x-rays and/or gamma rays, wherein said external source of photon therapy is selected from: external-beam radiation therapy, 2-D radiation therapy, systemic 3-dimensional conformal radiation therapy (3D-CRT), rotational, helical or arc-based intensity-modulated radiation therapy (IMRT), image-guided radiation therapy (IGRT), hypofractionated cone beam radiotherapy, tomotherapy, intra-operative radiotherapy (IORT), ultra-high dose rate (FLASH) radiotherapy, stereotactic radiosurgery or stereotactic body radiation therapy (SBRT or SABR, interchangeable throughout), such as via GammaKnife or CyberKnife or similar apparatuses.

In another embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, wherein said one or more doses of ionizing radiation selected from photon therapy or particle therapy is to be administered to the patient from an internal source comprising one or more particles or nanoparticles placed within or adjacent to said cancer, preferably wherein said one or more particles or nanoparticles placed within or adjacent to said cancer, emit free radicals, alpha-radiation, beta-radiation, x-rays or gamma rays, or combinations thereof, including hafnium oxide nanoparticles (NBTXR3, NanoBiotix, Inc), subsequently activated by radiotherapy.

In yet a further embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, wherein said one or more doses of ionizing radiation selected from photon therapy or particle therapy is to be administered to the patient from an internal source comprising one or more particles or nanoparticles placed within or adjacent to said cancer, preferably wherein said one or more particles or nanoparticles comprise brachytherapy seeds containing a radioisotope, such as phosphorus-32 (such as Oncosil), or iodine-125 (American SynQor, Inc).

Mechanistically, radiotherapy can trigger the production of pro-inflammatory chemokines including CXCL9, CXCL10 and CXCL16, resulting in the chemotactic recruitment of effector CD8+ T-cells into the TME (tumour microenvironment). Release of chemokines such as Mig and IP10 post-RT contribute to restoration of the vascular network and T-cell extravasation. Likewise, low-dose RT (e.g. 2 Gy) can reprogram tumour-resident macrophages from an immunosuppressive (‘M2’) phenotype to a TH1 polarizing iNOS+ (M1) phenotype.

In a preferred embodiment of the invention, one or more doses of ionizing radiation of particle therapy are administered to the patient. Published preclinical work supports the immunogenic potential of proton therapy and suggests that it may in fact have broader immunogenic applications than photons. For example, in vitro studies suggest that protons may mediate calreticulin translocation to cell surfaces at higher levels than photons, increasing cross-priming and sensitivity to CTLs. In vitro data has also shown that proton beam therapy (PBT) and X-ray irradiation achieves similar levels of survival of radiated melanoma cells, but only PBT induces long-term inhibition of migration. Anti-metastatic potential has also been demonstrated by PBT in human breast cancer cells and NSCLC cells, plus, a study in murine breast tumour (EMT6) cells and human salivary gland tumour cells showed that sublethal damage recovery was suppressed more after PBT than after X-ray irradiation. However, low energy proton beams induce tumor cell apoptosis through reactive oxygen species formation and activation of caspases, a process that may not be expected to prime CTLs through immunogenic cell death (ICD). In in vitro tumor cell models it has been shown that proton radiation, compared to photon radiation, resulted in a higher translocation of calreticulin thereby increasing the cross-priming of TAAa and the sensitivity of the tumor cells to CTL-mediated killing.

Moreover, protons, as charged particles, exhibit both dosimetric and biological differences in normal and cancer cells that may be able to not only enhance the immunoadjuvant effects of RT, but also reduce immunosuppressive mechanisms.

SABR presents several challenges despite improvements in technology, whereby grade 3+ toxicity remains an issue at higher doses and treatment efficacy is compromised by lower doses. Stereotactic MRI-guided adaptive radiation therapy (SMART) optimizes the accuracy and delivery of hypofractionated radiotherapy, allowing SABR to achieve higher doses while reducing damage to critical normal structures. This is achieved by respiratory gating, daily adaptation of the target volume, daily recontouring of organs at risk, daily reoptimisation of the treatment plan for the anatomy of the day, all of which lead to a smaller treatment volume and thus less dose to critical normal tissues. This may in turn allow for dose escalation to improve local control and results.

MR-guided radiotherapy is a novel treatment technique that has emerged in the past five years and presents promise for a variety of solid tumors. There are two commercially available MRI Linear accelerators (MR-linac) systems including one by ViewRay called MRldian (ViewRay Inc., Oakwood Village, Ohio) and a second by Elekta called Elekta Unity (Elekta AB, Stockholm, Sweden). In brief, rather than using a CT unit installed within a linear accelerator to localize the position of a tumor and normal organs prior to treatment delivery, a MR-linac combines an MRI device with a linear accelerator. Such a combination enables several capabilities that are uniquely helpful for the treatment of PDAC. First, MR-guidance offers improved soft tissue contrast and thereby the ability to distinguish the boundaries of different types of soft tissue. This can include the location of a tumor, small bowel, stomach, or vascular structures. Second, MR imaging on both commercially available MR linear accelerator devices is enabled when the beam is turned on and delivering radiotherapy. This results in the ability for normal organ movement to be tracked and monitored during the actual time of radiotherapy delivery. Such “real time” organ movement enables intra-treatment monitoring. Real time imaging will enable entirely novel tracking approaches, previously unappreciated. Third, with MRgRT at each fraction a new treatment plan can be generated based on the actual MRI visualized anatomy.

Currently, a very limited number of clinical trials using this technology are ongoing. The SMART trial is a well-known phase II trial examining the use of MR-guided radiation for locally advanced PDAC and is currently accruing (NCT03621644), in this study, patients are receiving 50 Gy fractionated over 5 days and administered on alternate days. A total of 133 patients are planned for enrollment into this multi-institutional trial. The primary endpoint of this study is grade 3 or higher gastrointestinal (GI) toxicity within 90 days of completion of radiotherapy. A second currently on-going study at Dana Farber Cancer Institute is a phase I/II study involving patients with either PDAC, lung cancer, or renal cancer (NCT04115254). The primary endpoint for the phase I portion of the study is delivery success rate for SMART across multiple tumor types.

In a preferred embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, wherein said one or more doses of ionizing radiation selected from photon therapy or particle therapy is to be administered to the patient using images or real-time guidance derived from fiducial seeds, X-rays, CT, PET, ultrasound or magnetic resonance (MR), wherein said images or real-time guidance is derived from magnetic resonance, preferably via MRI-guided LINAC or SBRT, such as MRldian or Elekta Unity.

In a preferred embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, wherein said one or more doses of ionizing radiation selected from photon therapy or particle therapy is to be administered to the patient using images or real-time guidance derived from fiducial seeds and said photon therapy comprises SBRT, such as CyberKnife.

In another embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, wherein said patient presents with (limited) metastases in lymph nodes and/or organs distant to the primary tumour, such as wherein said patient presents with between 1 and 10 oligometastatic tumours or lesions.

In another embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, wherein said patient presents with less than 5 oligometastatic tumours or lesions, as a total between the lung and the liver. Alternatively, said patient does not present with metastases in any lymph nodes and/or organs distant to the primary tumour.

In an embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, wherein said use comprises adjuvant, neoadjuvant or peri-adjuvant treatment or control of cancer comprising a primary tumour, in a patient intended to undergo, or having undergone, tumour resection surgery.

In an embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, wherein said one or more doses of ionizing radiation selected from photon therapy or particle therapy comprises a maximum biologically equivalent dose at alpha/beta of 10 Gray, (BED10) of greater than about 40 Gray, or greater than about 50 Gray, or greater than about 90 Gray.

In a preferred embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, wherein said one or more doses of ionizing radiation selected from photon therapy or particle therapy is to be administered to the patient as a single fraction or via a fractionated regimen. Said fractionated regimen may comprise between 2 and 50 fractions, such as between 3 and 10 fractions, preferably between 3 and 5 fractions.

In an embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, wherein the dose is between 40 and 50 Gray, administered in 5 fractions, suitably via MRI-guided LINAC or SBRT, such as MRldian or Elekta Unity, or conventional SBRT e.g. CyberKnife or GammaKnife. Alternatively, a dose range of 40 to 50 Gy may be applied, depending on Organ At Risk tolerance, together with dosimetric allowances, as known to those skilled in the art.

In an embodiment of the invention, the nominal dose is between about 40 and about 50 Gray, administered in 5 fractions, suitably via MRI-guided LINAC or SBRT, such as MRldian or Elekta Unity, or conventional SBRT e.g. CyberKnife or GammaKnife, preferably in 5 fractions of 8 Gy per fraction or 10 Gy per fraction, preferably where each fraction is administered on sequential days or alternate days.

The dose fractions of the ionizing radiation selected from photon therapy or particle therapy, preferably where an external source of photon therapy is selected from: external-beam radiation therapy, 2-D radiation therapy, systemic 3-dimensional conformal radiation therapy (3D-CRT), rotational, helical or arc-based intensity-modulated radiation therapy (IMRT), image-guided radiation therapy (IGRT), hypofractionated cone beam radiotherapy, tomotherapy, intra-operative radiotherapy (IORT), ultra-high dose rate (FLASH) radiotherapy, stereotactic radiosurgery or stereotactic body radiation therapy (SBRT or SABR, interchangeable throughout), such as via GammaKnife or CyberKnife or similar apparatuses. may be administered daily, or on alternate days or weekly or over longer periods, preferably SBRT (CyberKnife, GammaKnife, or similar) or MRI-guided SBRT, are administered on alternative days,

In an embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, wherein said one or more additional anticancer treatments or agents is selected from: adoptive cell therapy, surgical (tumour resection) therapy, chemotherapy, hormonal therapy, checkpoint inhibitor therapy, small molecule therapy such as metformin, receptor kinase inhibitor therapy, hyperthermia treatment, phototherapy, radiofrequency ablation therapy (RFA), anti-angiogenic therapy, cytokine therapy, cryotherapy, biological therapy, HDAC inhibitor e.g. OKI-179, BRAF inhibitor, MEK inhibitor, EGFR inhibitor, VEGF inhibitor, P13K delta inhibitor, PARP inhibitor, mTOR inhibitor, hypomethylating agents, oncolytic virus, TLR agonist including TLR2, 3, 4, 7, 8 or 9 agonists, such as rintatolimod, or TLR 5 agonists, MRx0518 (4D Pharma), STING agonists (including MIW815 and SYNB1891), mifamurtide and cancer vaccines such as GVAX or CIMAvax, and combinations thereof.

The TLR3 agonist polyriboinosinic:polyribocytidylic acid (poly I:C), induces type I IFN production as well as DC maturation. CD141+ DC are the human equivalents of murine CD8+/CD103+ DC and TLR3 and TLR8 are expressed by CD141+ DC. A most preferred TLR3 agonist for use in the invention is rintatolimod (Ampligen). Injection of mice with TLR3 and TLR7 agonists (resiquimod) results in upregulation of costimulatory molecules CD80, CD83 and CD86 by CD141+ and CD1c+ DC alike.

In further embodiments, said immunomodulator comprises a non-pathogenic, non-viable Mycobacterium which promotes CTL activity, wherein CTL activity includes the secretion of one or more pro-inflammatory cytokines and/or CTL mediated killing of target cells; and/or which promotes CD4+ T cell activation and/or CD4+ T cell proliferation and/or CD4+ T cell mediated cell depletion; and/or which promotes CD8+ T cell activation and/or CD8+ T cell proliferation and/or CD8+ T cell mediated cell depletion; and/or which enhances NK cell activity, and/or NK cell proliferation and/or NK cell mediated cell depletion, wherein enhanced NK cell activity includes increased depletion of target cells and/or pro-inflammatory cytokine release; and/or upregulation or stimulation of CD103+ CD141+ DCs; and/or which decreases or eliminates the differentiation, proliferation and/or activity of regulatory cells (Tregs), and/or the differentiation, proliferation, infiltration and/or activity of myeloid derived suppressor cells (MDSCs); and/or which decreases or eliminates the infiltration of inducible Tregs (iTregs) into a target site.

In an embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, wherein the cancer is selected from bladder cancer (including non-muscle invasive bladder cancer or urothelial cancer), prostate cancer, liver cancer, renal cancer, lung cancer, breast cancer, colorectal cancer, colon cancer, rectal cancer, pancreatic cancer, brain cancer (including glioblastoma), hepatocellular cancer, lymphoma, leukaemia, gastric cancer, cervical cancer, ovarian cancer, thyroid cancer, melanoma, head and neck cancer, skin cancer and soft tissue sarcoma and/or osteosarcoma, including pancreatic ductal adenocarcinoma (PDAC) and pancreatic neuroendocrine tumours (PNET), optionally wherein said cancer is metastatic.

In an embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, wherein, wherein the cancer is pancreatic cancer selected from: locally advanced pancreatic cancer (with or without nodal lesions), resectable pancreatic cancer, borderline resectable pancreatic cancer, checkpoint-refractory pancreatic cancer, chemotherapy-refractory pancreatic cancer, oligometastatic pancreatic cancer, pancreatic ductal adenocarcinoma (PDAC), and metastatic pancreatic ductal adenocarcinoma (mPDAC).

In an embodiment of the invention, the cancer is clinically defined by a TNM staging criteria, in which the patient has a primary tumour (T) of T1 to T4, and/or or a Node designation of NO, N1 or N2, or wherein said cancer is clinically defined as being Stage I, Stage II or Stage III or Stage IV, optionally wherein the patient has no evidence of metastasis (M0).

In another embodiment of the invention, the patient has undergone, or is intended to undergo, tumour resection surgery, optionally wherein the tumour resection surgery further comprises lymph node resection and optionally metastatic tumour resection when metastatic disease is present, such as metastatic pancreatic ductal adenocarcinoma (mPDAC).

In another embodiment of the invention, the cancer is determined as being microsatellite instability (MSI) high or MSI-low, as measured by a PCR-based assay and/or IHC staining for DNA mismatch repair deficient (dMMR) or proficient (pMMR) protein expression, preferably MSI-high and dMMR.

In a further embodiment of the invention, the patient is clinically classified as having a performance status of 0, 1, 1-, 2, 3 or 4 according to the ECOG scale, or classified as not being sufficiently fit to tolerate two or more chemotherapy regimens.

In a further embodiment of the invention, the patient also receives, or has previously received, one or more cytotoxic chemotherapeutic agents, such as FOLFIRINOX, mFOLFIRINOX, FOLFOX, NALIRIFOX, optionally wherein the patient demonstrated a partial response or stable disease following said FOLFIRINOX, mFOLFIRINOX, FOLFOX, or NALIRIFOX therapy.

In a further embodiment of the invention, the patient also receives, or has previously received nab-paclitaxel, suitably in combination with gemcitabine.

In a preferred embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, wherein the patient is also administered one or more checkpoint inhibitors, suitably selected from a cell, protein, peptide, antibody, ADC (antibody-drug conjugate), ISAC, Fab fragment (Fab), F(ab′)2 fragment, diabody, triabody, tetrabody, probody, fusion protein, single-chain variable region fragment (scFv), disulfide-stabilized variable region fragment (dsFv), or other antigen binding fragment thereof, which binds to the following receptors: CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1-R, CD94/NKG2A, TDO, TNFR, DcR3, CD27, CD28, CD40, CD122, CD137, OX40, GITR, ICOS and combinations thereof, preferably CTLA-4, PD-1 or PD-L1.

In a further preferred embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, wherein the one or more checkpoint inhibitors is selected from: ipilimumab, nivolumab, pembrolizumab, azetolizumab, BI 754091 (anti-PD-1), bavituximab (an IgG3 mab against PS), durvalumab, dostarlimab, tremelimumab, spartalizumab, avelumab, sintilimab, toripalimab, prolgolimab, tislelizumab, camrelizumab, MGA012 (retifanlimab), MGD013 (tebotelimab), MGD019, enoblituzumab, MGD009, MGC018, MEDI0680, miptenalimab (BI 754111, an anti-LAG-3), PDR001, FAZ053, TSR022, MBG453, relatlimab (BMS986016), LAG525 (IMP701), IMP321 (Eftilagimod alpha), REGN2810 (cemiplimab), REGN3767, pexidartinib (PLX3397), LY3022855, FPA008, BLZ945, GDC0919, epacadostat, emactuzumab (RG1755 targeting CSF-1 R), FPA150, indoximid, BMS986205, CPI-444, MEDI9447, PBF509, FS118 (bispecific for LAG-3 and PD-L1), lirilumab, Sym023, TSR-022, A2Ar inhibitors (e.g. EOS100850, AB928), NKG2A inhibitors such as monalizumab, and combinations thereof, optionally wherein the one or more checkpoint inhibitors is administered in a sub-therapeutic amount and/or duration.

In a further preferred embodiment of the invention, administration of said non-pathogenic non-viable Mycobacterium is prior to and/or after the administration of said one or more checkpoint inhibitors.

In one aspect of the present invention the non-pathogenic, non-viable Mycobacterium comprises a non-pathogenic heat-killed Mycobacterium. Examples of mycobacterial species for use in the present invention include M. vaccae, M. thermoresistibile, M. flavescens, M. duvalii, M. phlei, M. obuense, M. parafortuitum, M. sphagni, M. aichiense, M. rhodesiae, M. neoaurum, M. chubuense, M. tokaiense, M. komossense, M. aurum, M. w, M. tuberculosis, M. microti; M. africanum; M. kansasii, M. marinum; M. simiae; M. gastri; M. nonchromogenicum; M. terrae; M. triviale; M. gordonae; M. scrofulaceum; M. paraffinicum; M. intracellulare; M. avium; M. xenopi; M. ulcerans; M. diernhoferi, M. smegmatis; M. thamnopheos; M. flavescens; M. fortuitum; M. peregrinum; M. chelonei; M. paratuberculosis; M. leprae; M. lepraemurium and combinations thereof.

In a further preferred embodiment of the invention, the non-viable, non-pathogenic Mycobacterium is preferably selected from M. vaccae, including the strain deposited under accession numbers NCTC 11659 and associated designations such as SRL172, SRP299, IMM-201, DAR-901, and the strain as deposited under ATCC 95051 (Vaccae™), M. obuense, M. paragordonae (strain 49061), M. parafortuitum, M. paratuberculosis, M. brumae, M. aurum, M. indicus pranii, M.w, M. manresensis, M. kyogaense (as deposited under DSM 107316/CECT 9546), M. phlei, M. smegmatis, M. tuberculosis Aoyama B or H37Rv, RUTI, BCG, MTBVAC, VPM1002BC, SMP-105, mifamurtide or Z-100 and combinations thereof, preferably the strain of Mycobacterium obuense deposited under the Budapest Treaty under accession number NCTC 13365.

In a preferred embodiment of the invention, there is provided an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, wherein the non-pathogenic non-viable Mycobacterium is M. obuense NCTC 13365, preferably the rough variant and/or is heat-killed and/or presented as a fraction, fragment, sub-cellular component, lysate, homogenate, sonicate, or substantially in whole cell form.

In another most preferred embodiment, the whole cell or non-viable Mycobacterium is M. vaccae, including that deposited under NCTC 11569, or M. obuense, such as that deposited under NCTC 13365.

More preferably the whole cell, non-pathogenic non-viable Mycobacterium is a rough variant. Preferably, the whole cell, non-pathogenic non-viable Mycobacterium is heat-killed.

In preferred embodiments of the invention, the non-pathogenic, non-viable Mycobacterium is the rough variant, preferably the rough variant of M. obuense.

In other embodiments of the invention, the non-pathogenic non-viable Mycobacterium is the rough variant and/or whole cell, preferably the rough strain of Mycobacterium obuense deposited under the Budapest Treaty under accession number NCTC 13365.

In another embodiment of the invention, the non-pathogenic non-viable Mycobacterium has been inactivated by heat such as autoclaving, extended freeze drying, chemical exposure such as formaldehyde, or irradiation such as gamma irradiation or e-beam.

In another embodiment of the invention, the non-pathogenic non-viable Mycobacterium does not include BCG in live, attenuated form.

In a further embodiment, the non-pathogenic non-viable Mycobacterium is the rough variant and/or a presented as a fraction, fragment, sub-cellular component, lysate, homogenate, sonicate, or substantially in whole cell form.

In preferred embodiments of the invention, the non-pathogenic, non-viable Mycobacterium, suitably Mycobacterium obuense, is in a substantially whole cell form, such as where more than 50% or more of the mycobacteria in suspension are greater than 1 to 10 microns in diameter, as measured by laser diffraction (e.g. D50 value or mean particle size), or is in a form which has not been exposed to high pressure processing or other conditions to induce substantial cell lysis.

In another embodiment of the invention the immunomodulator is to be administered in a dose comprising 10³ to 10⁹ cells, or 0.0001 to 1.0 mg, preferably 0.05 mg to 1.0 mg.

In another embodiment of the invention, the immunomodulator is to be administered prior to tumour resection surgery and/or administration of said one or more additional anticancer treatments or agents, preferably checkpoint inhibitor therapy, for at least 1 to 3 doses.

In another embodiment of the invention, the immunomodulator is to be administered prior to tumour resection surgery and/or administration of said one or more additional anticancer treatments or agents, preferably checkpoint inhibitor therapy, for 3 doses.

In yet another embodiment of the invention, the immunomodulator is to be administered before and/or following tumour resection surgery or administration of said one or more additional anticancer treatments or agents, preferably checkpoint inhibitor therapy, optionally wherein said administration is over a period of 3 months, or 6 months, or 12 months or more.

In another embodiment of the invention, both the immunomodulator and said one or more additional anticancer treatments or agents, preferably checkpoint inhibitor therapy, are to be administered before and/or following tumour resection surgery, optionally wherein said administration of both the immunomodulator and said one or more additional anticancer treatments or agents, preferably checkpoint inhibitor therapy, is over a period of 3 months, or 6 months, or 12 months or more following said tumour resection surgery.

As would be understood by the skilled person, rough variants of M. obuense, for example, would lack cell surface-associated glycopeptidolipids (GPL) resulting in a characterised rough morphology with non-motile and non-biofilm-forming properties, as described in Roux et al. 2016, Open Biol 6: 160185. The amount of Mycobacterium administered to the patient in the present invention would be sufficient to elicit a protective immune response in the patient such that the patient's immune system would be able to mount an effective immune response. The amount of Mycobacterium administered to the patient is sufficient to elicit a protective immune response in the patient such that the patient's immune system is able to mount an effective immune response to the cancer or tumour.

In an embodiment of the invention, the effective amount of the immunomodulator composition elicits an immune response to the tumour antigens released from or following simultaneously, separately, or sequentially administering one or more doses of ionizing radiation selected from photon therapy or particle therapy to a patient.

In certain embodiments of the invention, there is provided a containment means comprising the effective amount of non-viable Mycobacterium for use in the present invention, which typically may be from 10³ to 10¹¹ organisms, preferably from 10⁴ to 10¹⁰ organisms, more preferably from 10⁶ to 10¹⁰ organisms, and even more preferably from 10⁶ to 10⁹ organisms. The effective amount of non-viable Mycobacterium for use in the present invention may be from 10³ to 10¹¹ organisms, preferably from 10⁴ to 10¹⁰ organisms, more preferably from 10⁶ to 10¹⁰ organisms, and even more preferably from 10⁶ to 10⁹ organisms. Most preferably the amount of non-viable whole cell Mycobacterium for use in the present invention is from 10⁷ to 10⁹ cells or organisms.

Typically, the composition according to the present invention may be administered at a dose of from 10⁸ to 10⁹ cells for human and animal use. Alternatively, the dose is from 0.0001 mg to 5 mg or 0.001 mg to 5 mg organisms, preferably 0.01 mg to 2 mg or 0.1 mg to 2 mg organisms, such as where the dose is approximately 0.5 mg or 1 mg or 0.5 mg or 1 mg organisms. The dose may be prepared as either a suspension or dry preparation.

M. vaccae and M. obuense induce a complex immune response in the host. Treatment with these preparations will stimulate innate and type-1 immunity, including Th1 and macrophage activation and cytotoxic cell activity. They also independently down-regulate inappropriate Th2 responses via immunoregulatory mechanisms. This restores the healthy balance of the immune system.

In certain embodiments of the invention, the amount of non-pathogenic non-viable Mycobacterium administered is between 0.0001 mg and 1 mg per unit dose, optionally wherein the unit dose is administered on two or more separate occasions separated by at least 7 days or more, such as administration on each of day 0, day 14 (+/−1, 3 or 5 days or more), and optionally day 30 (+/−5, 7 or 10 days or more) or day 45 (+/−7, 10 or 14 days or more).

In some embodiments, the amount of non-pathogenic non-viable Mycobacterium administered may be from 0.0001 mg to 1 mg per dose wherein the dose is administered 1, 2, 3, 4, 5, 6, 10 or 20 or more times over a number of days, weeks, or months, suitably wherein the Mycobacterium is M. obuense.

In other embodiments of the invention, the amount of non-pathogenic non-viable Mycobacterium administered may be from 0.0001 mg to 1 mg per dose, wherein the dose initially comprises one injection of 1 mg into one deltoid, or two injections of 0.5 mg in each deltoid, or two injections of 1.0 mg in each deltoid, followed by a second dose of either 0.5 or 1.0 mg 7 or 14 days or more later.

In certain embodiments of the invention, the amount of non-pathogenic non-viable Mycobacterium administered is between 0.0001 mg and 1 mg per unit dose, such as between about 0.5 and 1 mg, wherein the unit dose is administered on two or more separate occasions separated by at least 7 days or more, prior to tumour and/or nodal resection surgery, such as administration on each of day −35, day −21 and day −5 relative to (i.e. prior to) surgery, optionally where the −35 day dose is 1 mg, the −21 day dose is 0.5 mg and the −5 day dose is 0.5 mg. The checkpoint therapy, such as an anti-PD-L1 mab, suitably atezolizumab, pembrolizumab or cemiplimab, or as disclosed herein, may be administered on the same day or in between the unit dose of said Mycobacterium, such as day −28 and day −7, relative to (i.e. prior to) said surgery.

In a further embodiment of the invention, the amount of non-pathogenic non-viable Mycobacterium administered is between 0.0001 mg and 1 mg per unit dose, such as between about 0.5 and 1 mg, wherein the unit dose is administered on two or more separate occasions separated by at least 7 days or more, after tumour and/or nodal resection surgery, such as administration on each of day 21, day 35, day 49 et seq., following said surgery, optionally where the day 21 dose is 1 mg, the 35 day dose is 0.5 mg and the day 49 dose onward, is 0.5 mg. The checkpoint therapy, such as an anti-PD-L1 mab, suitably atezolizumab, pembrolizumab or cemiplimab, or as disclosed herein, may be administered on the same day or in between the unit dose of said Mycobacterium, such as day 28, day 42, day 56, et seq., following said surgery.

In yet another embodiment of the invention, the amount of non-pathogenic non-viable Mycobacterium administered is between 0.0001 mg and 1 mg per unit dose, such as between about 0.5 and 1 mg, wherein the unit dose is administered on two or more separate occasions separated by at least 7 days or more, prior to tumour and/or nodal resection surgery, such as administration on each of day −35, day −21 and day −5 relative to (i.e. prior to) surgery, optionally where the −35 day dose is 1 mg, the −21 day dose is 0.5 mg and the −5 day dose is 0.5 mg. The checkpoint therapy such as an anti-PD-L1 mab, suitably atezolizumab, or as disclosed herein, may be administered on the same day or in between the unit dose of said Mycobacterium, such as day −28 and day −7, relative to (i.e. prior to) said surgery, wherein a unit dose is administered on two or more separate occasions separated by at least 7 days or more, after tumour and/or nodal resection surgery, such as administration on each of day 21, day 35, day 49 et seq., following said surgery, optionally where the day 21 dose is 1 mg, the 35 day dose is 0.5 mg and the day 49 dose onward, is 0.5 mg. The checkpoint therapy such as an anti-PD-L1 mab, suitably atezolizumab or as disclosed herein, may be administered on the same day or in between the unit dose of said Mycobacterium, such as day 28, day 42, day 56, et seq., following said surgery. The present invention may be used to treat neoplastic disease, such as solid or non-solid cancers.

As used herein, “treatment” encompasses the prevention, reduction, control and/or inhibition of a neoplastic disease, including the regression or stabilization of a primary tumour and/or the regression or stabilization of one or metastases, or the prevention or inhibition of one or more metastases or micrometastases.

Neoplasia, tumours and cancers include benign, malignant, metastatic and non-metastatic types, and include any stage (I, II, III, IV or V) or grade (G1, G2, G3, etc.) of neoplasia, tumour, or cancer, or a neoplasia, tumour, cancer or metastasis that is progressing, worsening, stabilized or in remission. Preferably, the cancer at the onset of practising the invention is clinically defined as being Stage I, Stage II or Stage III or Stage IV.

In further embodiments, the non-pathogenic non-viable Mycobacterium is administered to the subject as a first line treatment, optionally simultaneously, separately or sequentially with administration of one or more therapeutic agents or cancer treatments, simultaneously, separately or sequentially with administration of one or more doses of ionizing radiation selected from photon therapy or particle therapy. A first line treatment may also be known as a primary treatment, and is typically administered to the subject or patient as a first treatment or therapy after initial diagnosis. Other therapeutic agents may include any pharmaceutical agents typically used to treat cancer, control cancer, provide pain relief or act prophylactically against any cancer-related pathologies, such as steroids, bisphosphonates and NSAIDs. Suitable hemotherapy may include administration of, but is not limited to, anti-neoplastics, anti-metabolites, microtubule inhibitors, nucleoside metabolic inhibitors and immunogenic agents. Preferred chemotherapeutic agents include FOLFIRINOX, Abraxane® (nab-paclitaxel), gemcitabine and pembrolizumab, preferably in multiple combinations. A nucleoside metabolic inhibitor may be administered, such as gemcitabine. Alternatively, a microtubule inhibitor may be administered, such as Abraxane (nab-paclitaxel).

In further embodiments, the non-pathogenic non-viable ell Mycobacterium is administered to the subject as a first or second line or maintenance treatment, simultaneously, separately or sequentially with administration of both gemcitabine and nab-paclitaxel, simultaneously, separately or sequentially with administration of one or more doses of ionizing radiation selected from photon therapy or particle therapy, optionally subsequent to FOLFIRINOX mFOLFIRINOX, FOLFOX, or NALIRIFOX therapy. This maintenance treatment is particularly applicable to patients with metastatic pancreatic ductal adenocarcinoma.

In a further embodiment, the non-pathogenic non-viable Mycobacterium is administered to the subject as a first or second line or maintenance treatment, simultaneously, separately or sequentially with administration of one or more doses of ionizing radiation selected from photon therapy or particle therapy, simultaneously, separately or sequentially with administration of both gemcitabine and one or more checkpoint inhibitors, suitably pembrolizumab, optionally subsequent to FOLFIRINOX, mFOLFIRINOX, FOLFOX, or NALIRIFOX therapy. This maintenance treatment is particularly applicable to patients with metastatic pancreatic ductal adenocarcinoma.

In further embodiments, the non-pathogenic non-viable whole cell Mycobacterium is administered to the subject as a first or second line or maintenance treatment, simultaneously, separately or sequentially with administration of one or more doses of ionizing radiation selected from photon therapy or particle therapy, simultaneously, separately or sequentially with administration of both gemcitabine and nab-paclitaxel, optionally subsequent to a partial response or stabilisation under FOLFIRINOX therapy. This maintenance treatment is particularly applicable to patients with metastatic pancreatic ductal adenocarcinoma.

In a further embodiment, the non-pathogenic non-viable whole cell Mycobacterium is administered to the subject as a first or second line or maintenance treatment, simultaneously, separately or sequentially with administration of one or more doses of ionizing radiation selected from photon therapy or particle therapy, simultaneously, separately or sequentially with administration of both gemcitabine and/or one or more checkpoint inhibitors, suitably pembrolizumab, optionally subsequent to an adequate response under FOLFIRINOX, mFOLFIRINOX, FOLFOX, or NALIRIFOX therapy. This maintenance treatment is particularly applicable to patients with metastatic pancreatic ductal adenocarcinoma.

In further embodiments, the non-pathogenic non-viable whole cell Mycobacterium is administered to the subject as a first or second line or maintenance treatment, simultaneously, separately or sequentially with administration of one or more doses of ionizing radiation selected from photon therapy or particle therapy simultaneously, separately or sequentially with administration of both gemcitabine and nab-paclitaxel, where said nab-paclitaxel is provided at a dose of 125 mg/m2 and administered as an i.v. infusion over about 30 minutes followed by Gemcitabine 1000 mg/m2 as a 30-minute i.v. infusion on D1, D8, D15 of a 28-day cycle. This maintenance treatment is particularly applicable to patients with metastatic pancreatic ductal adenocarcinoma.

The present invention may be used to treat a neoplastic disease, such as solid or non-solid cancers. Such diseases include a sarcoma, carcinoma, adenocarcinoma, melanoma, myeloma, blastoma, glioma, lymphoma or leukemia. Exemplary cancers include, for example, carcinoma, sarcoma, adenocarcinoma, melanoma, neural (blastoma, glioma), mesothelioma and reticuloendothelial, lymphatic or haematopoietic neoplastic disorders (e.g., myeloma, lymphoma or leukemia). In some aspects, a neoplasm, tumour or cancer includes a lung adenocarcinoma, lung carcinoma, diffuse or interstitial gastric carcinoma, colon adenocarcinoma, prostate adenocarcinoma, esophagus carcinoma, breast carcinoma, pancreas adenocarcinoma, ovarian adenocarcinoma, adenocarcinoma of the adrenal gland, adenocarcinoma of the endometrium or uterine adenocarcinoma. Neoplasia, tumours and cancers include benign, malignant, metastatic and nonmetastatic types, and include any stage (I, II, III, IV or V) or grade (G1, G2, G3, etc.) of neoplasia, tumour, or cancer, or a neoplasia, tumour, cancer or metastasis that is progressing, worsening, stabilized or in remission. The cancer at the onset of practising the invention is clinically defined as being Stage I, Stage II or Stage III or Stage IV or Stage V.

Cancers that may be treated according to the invention include but are not limited to, bladder, blood, bone, bone marrow, brain, breast, colon, esophagus, gastrointestines, gum, head, kidney, liver, lung, nasopharynx, neck, ovary, prostate, skin, stomach, testis, tongue, or uterus. Preferably, the cancer is selected from prostate cancer, liver cancer, renal cancer, lung cancer, breast cancer, colorectal cancer, pancreatic cancer, brain cancer, hepatocellular cancer, lymphoma, leukaemia, gastric cancer, cervical cancer, ovarian cancer, thyroid cancer, melanoma, head and neck cancer, skin cancer and soft tissue sarcoma and/or other forms of carcinoma. The tumour may be metastatic or a malignant tumour, most preferably metastatic. More preferably, the cancer is pancreatic, colorectal, prostate, ovarian cancer, most preferably the cancer is pancreatic, most preferably the cancer is metastatic pancreatic cancer.

The non-pathogenic non-viable whole cell Mycobacterium of the invention may be administered via the parenteral, oral, sublingual, nasal or pulmonary route. Further preferably, the parenteral route is selected from subcutaneous, intradermal, subdermal, intraperitoneal, intravenous, peritumoral, perilesional, intralesional or intratumoral, and combinations thereof. Suitably, intratumoral administration may be sequentially followed by intradermal administration.

In further embodiments, the or more additional anticancer agents are selected from: bevacizumab, cyclophosphamide, methotrexate, 5-fluorouracil, doxorubicin, mustine, vincristine, procarbazine, prednisolone, bleomycin, vinblastine, dacarbazine, etoposide, cisplatin, epirubicin, capecitabine, leucovorin, folinic acid, carboplatin, oxaliplatin, gemcitabine, FOLFIRINOX, modified FOLFIRINOX, FOLFOX, NALIRIFOX, paclitaxel, nab-paclitaxel, pemetrexed, irinotecan, temozolomide, an anti-PD-1 antibody, an anti-CTLA-4 antibody, an anti-PD-L1 antibody and/or an mTOR inhibitor and combinations thereof, wherein said one or more additional anticancer treatments or agents is administered intratumorally, intraarterially, intravenously, intravascularly, intrapleuraly, intraperitoneally, intratracheally, intranasally, pulmonarily, intrathecally, intramuscularly, endoscopically, intralesionally, percutaneously, subcutaneously, regionally, orally or by direct injection or perfusion.

In further embodiments, the or more additional anticancer agents are selected from: FOLFIRINOX, modified FOLFIRINOX, FOLFOX, NALIRIFOX, gemcitabine, nab-paclitaxel, an anti-PD-1 antibody, an anti-CTLA-4 antibody, an anti-PD-L1 antibody and/or an mTOR inhibitor, and combinations thereof, such as atezolizumab administered at a dose of either 1200 mg every three weeks or 840 mg every two weeks, or pembrolizumab administered at a dose of either 200 mg every three weeks or 400 mg every six weeks, or cemiplimab administered at a dose of 350 mg every three weeks.

In a further embodiment, the immunomodulator is initially to be administered prior to said one or more additional anticancer treatments or agents, optionally in conjunction with or after said one or more additional anticancer treatments or agents.

In yet a further embodiment, the treatment or control is monitored based on the measuring of one or more biomarkersm which may include any one or more of: prostate-specific antigen (PSA); carcinoembryonic antigen (CEA); CA19.9, PNR, INR, prognostic nutritional index (PNI); systemic immune-inflammation index (SII); and systemic inflammation score (SIS).

According to another aspect of the invention, the performance status of the subject stays the same or improves during and/or after said treatment or control of said cancer. In further embodiments, methods of the invention include, one or more of the following: 1) reducing or inhibiting growth, proliferation, mobility or invasiveness of tumour or cancer cells that potentially or do develop metastases, 2) reducing or inhibiting formation or establishment of metastases arising from a primary tumour or cancer to one or more other sites, locations or regions distinct from the primary tumour or cancer; 3) reducing or inhibiting growth or proliferation of a metastasis at one or more other sites, locations or regions distinct from the primary tumour or cancer after a metastasis has formed or has been established, 4) reducing or inhibiting formation or establishment of additional metastasis after the metastasis has been formed or established, 5) prolonged overall survival, 6) prolonged progression free survival, or 7) disease stabilisation.

A therapeutic benefit or beneficial effect is any objective or subjective, transient, temporary, or long-term improvement in the condition or pathology, or a reduction in onset, severity, duration or frequency of an adverse symptom associated with or caused by cell proliferation or a cellular hyperproliferative disorder such as a neoplasia, tumour or cancer, or metastasis. It may lead to improved survival. A satisfactory clinical endpoint of a treatment method in accordance with the invention is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of cell proliferation or a cellular hyperproliferative disorder such as a neoplasia, tumour or cancer, or metastasis. A therapeutic benefit or improvement therefore may be, but is not limited to destruction of target proliferating cells (e.g., neoplasia, tumour or cancer, or metastasis) or ablation of one or more, most or all pathologies, adverse symptoms or complications associated with or caused by cell proliferation or the cellular hyperproliferative disorder such as a neoplasia, tumour or cancer, or metastasis. However, a therapeutic benefit or improvement need not be a cure or complete destruction of all target proliferating cells (e.g., neoplasia, tumour or cancer, or metastasis) or ablation of all pathologies, adverse symptoms or complications associated with or caused by cell proliferation or the cellular hyperproliferative disorder such as a neoplasia, tumour or cancer, or metastasis. For example, partial destruction of a tumour or cancer cell mass, or a stabilization of the tumour or cancer mass, size or cell numbers by inhibiting progression or worsening of the tumour or cancer, can reduce mortality and prolong lifespan even if only for a few days, weeks or months, even though a portion or the bulk of the tumour or cancer mass, size or cells remain. Specific non-limiting examples of therapeutic benefit include a reduction in neoplasia, tumour or cancer, or metastasis volume (size or cell mass) or numbers of cells, inhibiting or preventing an increase in neoplasia, tumour or cancer volume (e.g., stabilizing), slowing or inhibiting neoplasia, tumour or cancer progression, worsening or metastasis, or inhibiting neoplasia, tumour or cancer proliferation, growth or metastasis.

In an embodiment of the invention, there is provided a non-pathogenic, non-viable Mycobacterium for use in the treatment, reduction, inhibition or control of cancer in a subject, simultaneously, separately or sequentially with administration of one or more doses of ionizing radiation selected from photon therapy or particle therapy, optionally wherein the subject is clinically classified as having a performance status of 0, 1, 1-, 2, 3 or 4 according to the ECOG Scale, or classified as not being sufficiently fit to tolerate two or more chemotherapy regimens, with the potential to elicit potent and durable immune responses with enhanced therapeutic benefit, preferably as measured by a decrease or stabilisation of tumour size of one or more said tumours, as defined by RECIST 1.1, or iRRC, or iRECIST, or irrRECIST, including stable diseases (SD), a complete response (CR) or partial response (PR) of the target tumour; and/or stable disease (SD) or complete response (CR) of one or more non-target tumours. Alternatively, therapeutic efficacy is assessed by Immune Related Response Criteria (irRC), iRECIST or irRECIST, as would be known to the skilled person.

In an embodiment of the invention, administration of the non-pathogenic non-viable Mycobacterium, simultaneously, separately or sequentially with administration of one or more doses of ionizing radiation selected from photon therapy or particle therapy, optionally with one or more checkpoint inhibitors, or FOLFIRINOX, mFOLFIRINOX, FOLFOX, NALIRIFOX, provides a detectable or measurable improvement or overall response according to the irRC (as derived from time-point response assessments and based on tumour burden), including one of more of the following: (i) irCR—complete disappearance of all lesions, whether measurable or not, and no new lesions (confirmation by a repeat, consecutive assessment no less than 4 weeks from the date first documented), (ii) irPR—decrease in tumour burden >50% relative to baseline (confirmed by a consecutive assessment at least 4 weeks after first documentation). Preferably, the checkpoint inhibitor employed is directed against CTLA-4, PD-1, or PD-L1, and combinations thereof.

In some embodiments of the invention, the treatment, reduction, inhibition or control of the early-stage or late-stage, advancer cancer further comprises administration of one or more additional anticancer treatments or agents.

The one or more additional anticancer treatments or agents may be selected from: adoptive cell therapy, surgical (tumour resection) therapy, chemotherapy, radiation therapy, hormonal therapy, checkpoint inhibitor therapy, small molecule therapy such as metformin, receptor kinase inhibitor therapy, hyperthermia treatment, phototherapy, radioablation therapy, radiofrequency ablation therapy (RFA), anti-angiogenic therapy, cytokine therapy, cryotherapy, biological therapy, HDAC inhibitor e.g. OKI-179, BRAF inhibitor, MEK inhibitor, EGFR inhibitor, VEGF inhibitor, P13K delta inhibitor, PARP inhibitor, mTOR inhibitor, hypomethylating agents, oncolytic virus, TLR agonist including TLR2, 3, 4, 7, 8 or 9 agonists such as rintatolimod, or TLR 5 agonists such as MRx0518 (4D Pharma), STING agonists (including MIW815 and SYNB1891), mifamurtide and cancer vaccines such as GVAX or CIMAvax, and combinations thereof.

In another preferred embodiment of the invention, the one or more doses of ionizing radiation selected from photon therapy or particle therapy results in damage in a tumour or tumour cells, so that the cells release antigens which are utilised by the immune system to recognize and target the tumour. The term includes ablative therapies. The release of tumour antigens can be shown by observing an increase in, for example, recall responses and cytotoxic T cell response.

Alternatively, the one or more doses of ionizing radiation selected from photon therapy or particle therapy results in damage in a tumour or tumour cells, so that the cells release antigens and/or there is: a) cell surface translocation of calreticulin (CRT), (b) extracellular release of HMGB1, and (c) extracellular release of adenosine-5′-triphosphate (ATP).

In another embodiment of the invention, the one or more additional anticancer treatments results in immunogenic cell death therapy, as described in WO2013/07998, incorporated by reference in its entirety. This therapy results in the induction of tumour immunogenic cell death, including apoptosis (type 1), autophagy (type 2) and necrosis (type 3), whereupon there is a release of tumour antigens that are able to both induce immune responses, including activation of cytotoxic CD8+ T cells and NK cells and to act as targets, including rendering antigens accessible to Dendritic Cells. The immunogenic cell death therapy may be carried out at sub-optimal levels, i.e. non-curative therapy such that it is not intended to fully remove or eradicate the tumour, but nevertheless results in some tumour cells or tissue becoming necrotic. The skilled person will appreciate the extent of therapy required in order to achieve this, depending on the technique used, age of the patient, status of the disease and particularly size and location of tumour or metastases Particularly preferred treatments include: microwave irradiation, embolisation, cryotherapy, ultrasound, high intensity focused ultrasound, cyberknife, hyperthermia, radiofrequency ablation, cryoablation, electrotome heating, hot water injection, alcohol injection, embolization, radiation exposure, photodynamic therapy, laser beam irradiation, and combinations thereof.

In another embodiment of the invention, the immunogenic cell death therapy or ionizing radiation may be carried out at sub-optimal levels, i.e. non-curative therapy such that it is not intended to fully remove or eradicate the tumour, but nevertheless results in some tumour cells or tissue.

In a further embodiment of the invention, the TLR agonists include MRx0518 (4D Pharma), mifamurtide (Mepact), Krestin (PSK), IMO-2125 (tilsotolimod), CMP-001, MGN-1703 (lefitolimod), entolimod, rintatolimod (Ampligen), SD-101, GS-9620, imiquimod, resiquimod, MEDI4736 (durvalumab), poly I:C, CPG7909, DSP-0509, VTX-2337 (motolimod), MEDI9197, NKTR-262, G100 or PF-3512676 and combinations thereof.

In a further embodiment of the invention, the chemotherapy comprises administration of one or more agents selected from: cyclophosphamide, methotrexate, 5-fluorouracil, doxorubicin, mustine, vincristine, procarbazine, prednisolone, bleomycin, vinblastine, dacarbazine, etoposide, cisplatin, epirubicin, capecitabine, leucovorin, folinic acid, carboplatin, oxaliplatin, gemcitabine, FOLFIRINOX, modified FOLIFIRINOX, FOLFOX, paclitaxel, pemetrexed, irinotecan temozolomide and combinations thereof.

In a further embodiment of the invention, the combination is suitable for treatment of pMMR-MSI-Low CRC tumours, i.e. mismatch repair proficient and microsatellite instability low tumours, such as combinations with or without FOLFOX and checkpoint inhibitors such as pemobrolizumab and/or atezolizumab, or durvalumab and/or tremelimumab, in combination with a Mycobacterium and administering one or more doses of ionizing radiation selected from photon therapy or particle therapy. Other combinations include Atezolizumab plus Bevacizumab together with a Mycobacterium and administering one or more doses of ionizing radiation selected from photon therapy or particle therapy.

In a further embodiment of the invention, the combination is suitable for treatment of pMMR-MSI-Low CRC tumours, wherein the non-pathogenic, non-viable Mycobacterium is administered in combination with one or more doses of ionizing radiation selected from photon therapy or particle therapy, together with one or more of the following combinations: RFA and/or EBRT and/or pembrolizumab; RFA and/or durvalumab and/ortremelimumab; atezolizumab and/or bevacizumab and/or FOLFOX or mFOLFIRINOX; atezolizumab and cobimetinib; nivolumab+ipilimumab+cobimetinib; atezolizumab+cobimetinib+bevacizumab; Atezolizumab+CEA-BTC antibody; Pembrolizumab+indoximod; nivolumab+epacadostat; durvalumab+pexidartinib.

In a further embodiment of the invention, the combination administering one or more doses of ionizing radiation selected from photon therapy or particle therapy, together with either radiofrequency ablation (RFA) or external beam radiation therapy (EBRT) in patients with CRC, together with checkpoint inhibitors such as pemobrolizumab and/or atezolizumab, or durvalumab and/or tremelimumab, in combination with a Mycobacterium.

In a further embodiment of the invention, the adjuvant, neoadjuvant or peri-adjuvant therapy relative to tumour resection as disclosed herein is applied to patients with stage III dMMR and/or MSI-High colorectal cancer, or colon cancer or rectal cancer, wherein said patient optionally presents with an ECOG Performance Status of 0 (PS0), 1 (PS1) or PS1-, or 2 (PS2).

In a further embodiment of the invention, the adjuvant, neoadjuvant or peri-adjuvant therapy relative to tumour resection, as disclosed herein, is applied to patients who present with an ECOG Performance Status of 0 (PS0), 1 (PS1) or PS1- or 2 (PS2) and wherein said Performance Status value is the same or improved when assessed post-surgery or at the end of the inventive combination therapy.

In a further embodiment of the invention, the adjuvant, neoadjuvant or peri-adjuvant therapy disclosed herein is applied to patients with stage III pMMR/MSI-Low colorectal cancer, or colon cancer or rectal cancer i.e. mismatch repair proficient and microsatellite instability low tumours in patients with colorectal cancer, or colon cancer or rectal cancer, wherein said patient presents with an ECOG Performance Status of 0 (PS0), 1 (PS1) or PS1- or 2 (PS2).

In some preferred embodiments, the one or more additional anticancer treatments or agents may include checkpoint inhibitor agents. Such checkpoint inhibitors may be selected from: ipilimumab, nivolumab, pembrolizumab, azetolizumab, BI 754091 (anti-PD-1), bavituximab (an IgG3 mab against PS), durvalumab, dostarlimab, tremelimumab, spartalizumab, avelumab, sintilimab, toripalimab, prolgolimab, tislelizumab, camrelizumab, MGA012 (retifanlimab), MGD013 (tebotelimab), MGD019, enoblituzumab, MGD009, MGC018, MED10680, miptenalimab (BI 754111, an anti-LAG-3), PDR001, FAZ053, TSR022, MBG453, relatlimab (BMS986016), LAG525 (IMP701), IMP321 (Eftilagimod alpha), REGN2810 (cemiplimab), REGN3767, pexidartinib (PLX3397), LY3022855, FPA008, BLZ945, GDC0919, epacadostat, emactuzumab (RG1755 targeting CSF-1 R), FPA150, indoximid, BMS986205, CPI-444, MEDI9447, PBF509, FS118 (bispecific for LAG-3 and PD-L1), lirilumab, Sym023, TSR-022, A2Ar inhibitors (e.g. EOS100850, AB928), NKG2A inhibitors such as monalizumab, and combinations thereof, optionally wherein the one or more checkpoint inhibitors is administered in a sub-therapeutic amount and/or duration.

In other embodiments of the invention, the tumour and/or metastases may be surgically removed via resection, which results in a tumour margin as defined according to the AJCC 8^{th} Edition, such as an R0 resection margin, where no cancer cells are seen microscopically at the primary tumour site; or R1, where cancer cells are present microscopically at the primary tumour site; or, R2, where macroscopic residual tumour is found at the primary cancer site and/or regional lymph nodes.

In a further embodiment, a combination according to the invention is administered within 1, 2, 5, 10, 20, 30, 40, 50, 60 or 70 days before, and/or after tumour resection, wherein said tumour resection is suitably an R0, R1 or R2 resection, preferably R0 and no later than 70 days after.

Administration of the Mycobacterium may be administered intradermally, initially at a dose of 1.0 mg before and/or following tumour resection, optionally followed by administration of the Mycobacterium at a dose of 0.5 mg or 1 mg every 2 weeks for one month, followed by a dose of 0.5 mg or 1 mg Mycobacterium every 4 weeks for 11 months or more, preferably wherein said Mycobacterium is M. obuense NCTC 13365.

In a further embodiment, the invention provides an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium wherein said non-viable, non-pathogenic Mycobacterium and one or more additional anticancer treatments or agents are to be administered, optionally at the same or different time, via the same or different route of administration, and where the patient demonstrates a pathological complete or partial/subtotal response or tumour regression at 5 or 6 or 12 weeks or later post-surgery or end of therapy, and/or an increased disease-free survival (DFS) or Overall Survival (OS) at 1, 2, 3 or 5 years or later, post-surgery or end of therapy, and/or an improved quality of life (assessed by EORTC QLQ-C30 and PRO-CTCAE questionnaires), and/or no detectable ctDNA at 12 months or later, post-surgery or end of therapy, preferably wherein said Mycobacterium is M. obuense NCTC 13365.

Circulating tumor DNA (“ctDNA”) is found in the bloodstream and refers to DNA that comes from cancerous cells and tumours. Measurement of ctDNA has emerged as a promising blood-based biomarker for monitoring disease status of patients with advanced cancers. The presence of ctDNA in the blood is a result of biological processes, namely tumour cell apoptosis and/or necrosis, and can be used to monitor different cancers by targeting cancer-specific mutation.

An invention method may not take effect immediately. For example, treatment may be followed by an increase in the neoplasia, tumour or cancer cell numbers or mass, but over time eventual stabilization or reduction in tumour cell mass, size or numbers of cells in a given subject may subsequently occur. Additional adverse symptoms and complications associated with neoplasia, tumour, cancer and metastasis that can be inhibited, reduced, decreased, delayed or prevented include, for example, nausea, lack of appetite, lethargy, pain and discomfort. Thus, a partial or complete decrease or reduction in the severity, duration or frequency of an adverse symptom or complication associated with or caused by a cellular hyperproliferative disorder, an improvement in the subject's quality of life and/or well-being, such as increased energy, appetite, psychological well-being, are all particular non-limiting examples of therapeutic benefit.

A therapeutic benefit or improvement therefore can also include a subjective improvement in the quality of life of a treated subject. In an additional embodiment, a method prolongs or extends lifespan (survival) of the subject. In a further embodiment, a method improves the quality of life of the subject. In another embodiment, administration of the checkpoint inhibitor results in a clinically relevant improvement in one or more markers of disease status and progression selected from one or more of the following: (i): overall survival, (ii): progression free survival, (iii): overall response rate, (iv): reduction in metastatic disease, (v): circulating levels of tumour antigens such as carbohydrate antigen 19.9 (CA19.9) and carcinembryonic antigen (CEA) or others depending on tumour, (vii) nutritional status (weight, appetite, serum albumin) and a reduction in loss of lean body mass i.e. prevention or reduction in cachexia, (viii): pain control or analgesic use, (ix): CRP/albumin ratio.

Treatment with non-pathogenic, non-viable M. vaccae and/or M. obuense gives rise to more complex immunity including not only the development of innate immunity and type-1 immunity, but also immunoregulation which more efficiently restores appropriate immune functions.

In a further preferred embodiment, the checkpoint inhibitor is an antibody selected from the group consisting of: ipilimumab, nivolumab, pembrolizumab, azetolizumab, tremelimumab, cemiplimab, avelumab camrelizumab or relatlimab, or combinations thereof.

In another embodiment, the checkpoint inhibitor is pembrolizumab and said subject or patient has mismatch repair-deficient tumours associated with said cancer and/or exhibits PD-L1 expression in at least 5%, 10%, or 20%, or 30%, or 40%, or 50% or more of tumour cells, as measured using the SP142 immunohistochemistry antibody assay.

In a further embodiment, the checkpoint inhibitor is ipilimumab and is administered intravenously at a dose of 10, 5, or 2 mg/mg or less every three weeks, optionally for a maximum of four infusions. In another embodiment, the checkpoint inhibitor is nivolumab and is administered intravenously at a dose of at least 1, 2, 3, or 5 or 10 mg/mg or more every four weeks, optionally wherein said patient exhibits PD-L1 expression in at least 1%, 5% or 10% or more of tumour cells, as measured using the SP142 immunohistochemistry antibody assay.

In a further embodiment, the checkpoint inhibitor is azetolizumab and is administered intravenously at a dose of about 1, 5 or 10 or 15 mg/kg or more every three weeks, optionally wherein said patient exhibits PD-L1 expression in at least 1%, 5% or 10% or more of tumour cells and/or tumour-infiltrating immune cells selected from B-cells and NK cells, as measured using the SP142 immunohistochemistry antibody assay. Alternatively, atezolizumab is administered as an 840 mg or 1200 mg infusion.

The term “combination” as used throughout the specification, can encompass the administration of the one or more doses of ionizing radiation selected from photon therapy or particle therapy to the patient, simultaneously, separately or sequentially with administration of the, non-pathogenic non-viable Mycobacterium, in a patient that has undergone, or is intended to undergo tumour resection surgery and/or administration of one or more additional anticancer treatments and/or agents Accordingly, the of one or more additional anticancer treatments and/or agents and the non-pathogenic non-viable Mycobacterium may be present in the same or separate pharmaceutical formulations, and administered at the same time or at different times as the one or more doses of ionizing radiation. Alternatively, the non-pathogenic whole cell non-viable Mycobacterium and the one or more additional anticancer treatments and/or agents may be provided as separate medicaments for administration at the same time or at different times. Preferably, the non-pathogenic non-viable Mycobacterium and one or more additional anticancer treatments and/or agents, such as a checkpoint inhibitor, are provided as separate medicaments for administration at different times. When administered separately and at different times, either the non-pathogenic non-viable Mycobacterium or one or more additional anticancer treatments and/or agents, such as a checkpoint inhibitor may be administered first, however, it is suitable to administer one or more additional anticancer treatments and/or agents, such as a checkpoint inhibitor followed by the non-pathogenic non-viable Mycobacterium. In each of the above scenarios, the one or more doses of ionizing radiation selected from photon therapy or particle therapy, may be administered before, during or after said non-pathogenic non-viable Mycobacterium and/or one or more additional anticancer treatments and/or agents. In addition, either can be administered on the same day or at different days, and they can be administered using the same schedule or at different schedules during the treatment cycle. In an embodiment of the invention, a treatment cycle consists of the administration of a non-pathogenic non-viable Mycobacterium daily, weekly fortnightly or monthly, simultaneously with one or more additional anticancer treatments and/or agents e.g. checkpoint inhibitor, weekly, fortnightly or monthly.

In one embodiment, the non-pathogenic non-viable Mycobacterium is administered to the patient before and after said one or more additional anticancer treatments and/or agents e.g. checkpoint inhibitor. Dose delays and/or dose reductions and schedule adjustments are performed as needed depending on individual patient tolerance to treatments. Alternatively, the administration of one or more additional anticancer treatments and/or agents e.g. checkpoint inhibitor may be performed simultaneously with the administration of the effective amounts of the non-pathogenic non-viable Mycobacterium.

In an aspect of the invention, the effective amount of the non-pathogenic non-viable Mycobacterium may be administered as a single dose. Alternatively, the effective amount of the non-pathogenic non-viable Mycobacterium may be administered in multiple (repeat) doses, for example two or more, three or more, four or more, five or more, ten or more, or twenty or more repeat doses. The non-pathogenic non-viable Mycobacterium may be administered between about 4 weeks and about 1 day prior to one or more additional anticancer treatments and/or agents e.g. checkpoint inhibitor and/or prior to or one or more doses of ionizing radiation selected from photon therapy or particle therapy, may be administered before, during or after said non-pathogenic non-viable Mycobacterium or one or more additional anticancer treatments and/or agents, such as between about 4 weeks and 1 week, or about between 3 weeks and 1 week, or about between 3 weeks and 2 weeks before. Administration may be presented in single or multiple doses.

In one embodiment of the present invention, the non-pathogenic non-viable Mycobacterium may be in the form of a medicament administered to the patient in a dosage form. A container according to the invention in certain instances may be a vial, an ampoule, a syringe, a pre-filled syringe, a capsule, tablet or a tube. In some cases, the mycobacteria may be lyophilized and formulated for resuspension prior to administration. However, in other cases, the mycobacteria are suspended in a volume of a pharmaceutically acceptable liquid. In some embodiments, there is provided a container comprising a single unit dose of mycobacteria suspended in pharmaceutically acceptable carrier wherein the unit dose comprises about 1×10⁶ to about 1×10¹⁰ killed organisms. In some embodiments the liquid comprising suspended mycobacteria is provided in a volume of between about 0.05, or 0.1 ml and 10 ml, or between about 0.3 ml and 2 ml or between about 0.5 ml and 2 ml. The foregoing compositions provide ideal units for immunotherapeutic applications described herein.

Embodiments discussed in the context of a methods and/or composition of the invention may be employed with respect to any other method or composition described herein. Thus, an embodiment pertaining to one method or composition may be applied to other methods and compositions of the invention as well. In some cases, non-pathogenic non-viable Mycobacteria are administered to specific sites on or in a subject. For example, the mycobacterial compositions according to the invention, such as those comprising M. obuense in particular, may be administered into or adjacent to tumours or into or adjacent to lymph nodes, such as those that drain tissue surrounding a tumour. Thus, in certain instances sites administration of mycobacterial composition may be into or near the posterior cervical, tonsillar, axillary, inguinal, anterior cervical, sub-mandibular, sub mental or superclavicular lymph nodes. The non-pathogenic heat-killed Mycobacterium may be administered for the length of time the cancer or tumour(s) is present in a patient or until such time the cancer, e.g. the primary tumour and/or one or more metastases, has regressed and/or stabilized and/or been resected. The non-pathogenic non-viable Mycobacterium may also be continued to be administered to the patients once the cancer or tumour or metastases has regressed or stabilised or been resected.

In another particularly preferred embodiment, the immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, wherein said immunomodulator comprises non-pathogenic non-viable Mycobacterium, results in an abscopal effect.

Ionizing radiation can reduce tumour growth outside the field of radiation, known as the abscopal effect, from the Latin “ab scopus”, “away from the target”. Although it has been reported in multiple malignancies, the abscopal effect remains a rare and poorly understood event. Their rare occurrence reflects the fact that, by itself, standard radiotherapy is inadequate at subverting the existing immuno-suppression characteristic of the microenvironment of an established tumour. An abscopal effect is defined as a measurable response in any of the measurable lesions outside the radiation field, as assessed by e.g. PET-CT, or stabilization or regression of distant metastases, relative to the irradiated tumour(s).

Mycobacterial compositions according to the invention will comprise an effective amount of mycobacteria typically dispersed in a pharmaceutically acceptable carrier. The phrases “pharmaceutically or pharmacologically acceptable” refers to molecular entities and compositions that do not produce an adverse, allergic or other untoward reaction when administered to an animal, such as, for example, a human, as appropriate. The preparation of a pharmaceutical composition that contains mycobacteria will be known to those of skill in the art. Moreover, for animal (e.g., human) administration, it will be understood that preparations should meet sterility, pyrogenicity, general safety and purity standards. A specific example of a pharmacologically acceptable carrier as described herein is borate buffer or sterile saline solution (0.9% NaCl). As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavouring agents, dyes, such like materials and combinations thereof, as would be known to one of ordinary skill in the art.

In another embodiment, the non-pathogenic non-viable Mycobacterium is administered via a parenteral route selected from subcutaneous, intradermal, subdermal, intraperitoneal, intravenous and intravesicular injection. Intradermal injection enables delivery of an entire proportion of the mycobacterial composition to a layer of the dermis that is accessible to immune surveillance and thus capable of electing an anti-cancer immune response and promoting immune cell proliferation at local lymph nodes. In some embodiments of the invention mycobacterial compositions are administered by direct intradermal injection, it is also contemplated that other methods of administration may be used in some case. Thus in certain instances, the non-pathogenic non-viable Mycobacterium of the present invention can be administered by injection, infusion, continuous infusion, intravenously, intradermally, intraarterially, intraperitoneally, intralesionally, intravitreally, intravaginally, intrarectally, topically, intratumourally, intramuscularly, intraperitoneally, subcutaneously, subconjunctival, intravesicularlly, mucosally, intrapericardially, intraumbilically, intraocularally, orally, intracranially, intraarticularly, intraprostaticaly, intrapleural, intratracheally, intranasally, intranodally, topically, locally, inhalation (e.g. aerosol inhalation), perilesionally, peritumorally, percutaneously, regionally, stereotactically, orally or by direct injection or perfusion via a catheter, via a lavage, or by other method or any combination of the forgoing as would be known to one of ordinary skill in the art.

A further embodiment of the invention is a method of treating, reducing, inhibiting or controlling cancer in a subject, wherein said method comprises simultaneously, separately or sequentially administering to the subject (i) one or more therapeutic agents and/or cancer treatments and (ii) a non-pathogenic non-viable whole cell Mycobacterium, wherein said method results in enhanced therapeutic efficacy relative to administration of one or more therapeutic agents and/or cancer treatments alone, and wherein the subject is clinically classified as having a performance status of 2, 3 or 4 according to the ECOG Scale, or as not being sufficiently fit to tolerate two or more chemotherapeutic regimens. Preferably, the subject is classified as having a performance status of 0, 1 or 2, or suitably 0 or 1.

In other aspects of the invention, the non-pathogenic non-viable Mycobacterium used in the method is M. vaccae, M. parafortuitum, M. aurum, M. indicus pranii and combinations thereof, most preferably M. obuense. Preferably, the non-pathogenic non-viable Mycobacterium is heat-killed. Preferably, the non-pathogenic non-viable Mycobacterium is the rough variant. Further preferably, the non-viable whole cell Mycobacterium is administered to the subject as a first line treatment, optionally simultaneously, separately or sequentially with administration of one or more therapeutic agents or modalities.

In some aspects of the method, the subject or patient receives one or more cytotoxic chemotherapeutic agents, such as FOLFIRINOX. The subject may also be administered a nucleoside metabolic inhibitor, preferably gemcitabine. Preferably, the subject may be administered a microtubule inhibitor, most preferably nab-paclitaxel, optionally in combination with gemcitabine. In some aspects of the method, the subject may also be administered targeted radiotherapy, such as stereotactic body radiation therapy (e.g. Cyberknife®).

In some aspects of the invention, the subject is also administered one or more checkpoint inhibitors, selected from a cell, protein, peptide, antibody ADC (antibody-drug conjugate), Fab fragment (Fab), F(ab′)2 fragment, diabody, triabody, tetrabody, probody, single-chain variable region fragment (scFv), disulfide-stabilized variable region fragment (dsFv), or other antigen binding fragment thereof, directed against CTLA-4, PD-1, PD-L1, PD-L2, B7-H3, B7-H4, B7-H6, A2AR, IDO, TIM-3, BTLA, VISTA, TIGIT, LAG-3, CD40, KIR, CEACAM1, GARP, PS, CSF1 R, CD94/NKG2A, TDO, TNFR, DcR3, CD27, CD28, CD40, CD122, CD137, OX40, GITR, ICOS and combinations thereof. In some aspects of the method, the one or more checkpoint inhibitors can be administered in a sub-therapeutic amount and/or duration. In some aspects of the invention, the administration of said non-pathogenic non-viable whole cell Mycobacterium is prior to and/or after the administration of the checkpoint inhibitor.

In an embodiment of the invention, the co-stimulatory checkpoint therapy comprises administration of one or more binding agents selected from utomilumab, urelumab, MOXR0916. PF04518600, MED10562, GSK3174988, MED16469. R07009789, CP870893, BMS986156, GWN323, JTX-2011, varlilumab, MK-4166, NKT-214 and combinations thereof.

Suitable specific combinations include: durvalumab+tremelimumab, nivolumab+ipilimumab, pembrolizumab+ipilimumab, MEDI0680+durvalumab, PDR001+FAZ053, mivolumab+ TSR022, PDR001+MBG453, nivolumab+BMS 986016, PDR001+LAG525, Pembrolizumab+IMP321, REGN2810 (cemiplimab)+REGN3767, and other suitable combinations.

In some embodiments, the checkpoint inhibitor therapy may further comprise co-stimulatory checkpoint therapy, directed against any one of the following combinations: CTLA-4 and CD40, CTLA-4 and OX40, CTLA-4 and IDO, OX-40 and PD-L1, PD-1 and OX-40, CD27 and PD-L1, PD-1 and CD137, PD-L1 and CD137, OX-40 and CD137, CTLA-4 and IDO, PD-1 and IDO, PD-L1 and IDO, PD! And A2AR, PD-L1 and A2AR, PD1 and GITR, PD-L1 and GITR, PD1 and ICOS, PD-L1 and ICOS, PD1 and CD27, PD-L1 and CD27, PD1 and CD122, PD-L1 and CD122, PD1 and CSF1R, PD-L1 and CSF1R, and other such suitable combinations.

Suitable specific combinations include: Avelumab+utomilumab, Nivolumab+urelumab, Pembrolizumab+utomilumab, Atezolimumab+MOXR0916±bevacizumab, Avelumab+PF-04518600, Durvalumab+MED10562, Pembrolizumab+GSK3174998, Tremelimumab+durvalumab+MEDI6469, Tremelimumab+MED10562, Utomilumab+PF-04518600, Atezolimumab+R07009789, Tremelimumab+CP870893, Nivolumab+BMS986156, PDR001+GWN323, Nivolumab+JTX-2011, Atezolizumab+GDC0919, Ipilimumab+epacadostat, Ipilimumab+indoximid, Nivolumab+BMS986205, Pembrolizumab+epacadostat, Atezolizumab+CPI-444, Durvalumab+MEDI9447, PDR001+PBF509, Nivolumab+varlilumab, Atezolizumab+varlilumab, Nivolumab+NKTR-214, Durvalumab+Pexidartinib (PLX3397), Durvalumab+LY3022855, Nivolumab+FPA008, Pembrolizumab+Pexidartinib, PDR001+BLZ945, Tremelimumab+LY3022855.

In a further embodiment, the checkpoint inhibitor therapy comprises administration of a blocking agent, wherein said blocking agent is an antibody selected from the group consisting of: AMP-224 (Amplimmune, Inc), BMS-986016 (anti-LAG-3 mab called relatlimab) or MGA-271 (anti-B7-H3 mab called enoblituzumab), and combinations thereof. AMP-224, also known as B7-DCIg, is a PD-L2-Fc fusion soluble receptor described in WO2010/027827 and WO2011/066342. Another suitable agents includes bintrafusp alfa, a bifunctional fusion protein designed to block the TGF-β and PD-L1 receptors.

In a further embodiment, the checkpoint inhibitor therapy comprises administration of a blocking agent wherein said blocking agent is an antibody that specifically binds to B7-H3 such as enoblituzumab, an engineered Fc humanized IgG1 monoclonal antibody against B7-H3 with potent anti-tumor activity (Macrogenics, Inc.), or MGD009, a B7-H3 dual affinity re-targeting (DART) protein that bind both CD3 on T cells and B7-H3 on the target cell which has been found to recruit T cells to the tumour site and promote tumour 10 eradication, or MGD009 is a humanized DART protein. MGCO18 is anti-B7-H3 antibody drug conjugate (ADC) with a duocarmycin payload and cleavable peptide linker.

In some embodiments, the checkpoint inhibitor therapy comprises administration of an anti-B7-H3-binding protein selected from the group consisting of DS-5573 (Daiichi Sankyo, Inc.), enoblituzumab (MacroGenics, Inc.), and omburtamab [8H9] (Y-mabs Therapeutics, Inc), an antibody against B7-H3 labeled with radioactive iodine (I-131), and combinations thereof.

In some embodiments, the checkpoint inhibitor therapy comprises administration of indoleamine-2,3-dioxygenase (IDO) inhibitors such as D-I-methyl-tryptophan (Lunate) and other compounds described in U.S. Pat. No. 7,799,776, the contents of which are incorporated herein by reference.

In certain embodiments, the co-stimulatory checkpoint therapy upregulates the cellular immune system, wherein said co-stimulatory checkpoint therapy comprises administration of a binding agent, selected from a cell, protein, peptide, antibody or antigen binding fragment thereof, directed against CD27, OX40, GITR, or CD137, and combinations thereof, such as CD137 agonists including without limitation BMS-663513 (urelumab, an anti-CD137 humanized monoclonal antibody agonist, Bristol-Myers Squibb); agonists to CD40, such as CP-870,893 (a-CD40 humanized monoclonal antibody, Pfizer); OX40 (CD 134) agonists (e.g. anti-OX40 humanized monoclonal antibodies, AgonOx and those described in U.S. Pat. No. 7,959,925), and Astra Zeneca's MED10562, a humanised OX40 agonist; MED16469, murine OX40 agonist; and MED16383, an OX40 agonist; or agonists to CD27 such as CDX-1127 (a-CD27 humanized monoclonal antibody, Celldex). Suitable anti-GITR antibodies include TRX518 (Tolerx), MK-1248 (Merck), CK-302 and suitable anti-4-1BB antibodies for use in the invention include PF-5082566 (Pfizer).

In one embodiment of the invention, the subject is further administered one or more nutraceuticals selected from amino acids, antioxidants, fats, vitamins, trace elements, minerals, micronutrients, plant extracts, phytochemicals, fibres, prebiotics, probiotics, and/or a combination thereof, preferably vitamin D, vitamin C and/or zinc. Vitamin D may have a pleiotropic effect in immune cells, including macrophages, and an immunomodulatory role of vitamin D in various immune cells and diseases has been demonstrated. Accordingly, suitable presentations of vitamin D may include oral or injected formulations of paricalcitol e.g. 1 mcg orally; cholecalciferol, 60,0001U orally per week or single injections of 300,000 IUs; or 300,000 IUs of ergocalciferol injected.

In addition, the cancer may specifically be of the following histological type, though it is not limited to the following: neoplasm, malignant carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumour, malignant bronchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma with squamous metaplasia; thymoma, malignant ovarian stromal tumour, malignant thecoma, malignant granulosa cell tumour, malignant androblastoma, malignant Sertoli cell carcinoma; Leydig cell tumour, malignant lipid cell tumour, malignant paraganglioma, malignant extra-mammary paraganglioma, malignant pheochromocytoma; glomangiosarcoma; malignant melanoma; amelanotic melanoma; superficial spreading melanoma; malignant melanoma in giant pigmented nevus; metastatic melanoma, Lentigo Maligna, Lentigo Maligna Melanoma, cutaneous squamous cell carcinoma, Nodular Melanoma, Acral Lentiginous Melanoma, desmoplastic Melanoma, epithelioid cell melanoma; blue nevus, malignant sarcoma; fibrosarcoma; fibrous histiocytoma, malignant myxosarcoma; liposarcoma; leiomyosarcoma; rhabdomyosarcoma; embryonal rhabdomyosarcoma; alveolar rhabdomyosarcoma; undifferentiated pleomorphic sarcoma; stromal sarcoma; mixed tumour; Mullerian mixed tumour; nephroblastoma; hepatoblastoma; carcinosarcoma; mesenchymoma, malignant Brenner tumour, malignant phyllodes tumour, malignant synovial sarcoma; mesothelioma, malignant dysgerminoma; embryonal carcinoma; teratoma, malignant; struma ovarii, malignant; choriocarcinoma; mesonephroma, malignant hemangiosarcoma; hemangioendothelioma, malignant Kaposi's sarcoma; hemangiopericytoma, malignant lymphangiosarcoma; osteosarcoma; juxtacortical osteosarcoma; chondrosarcoma; chondroblastoma, malignant mesenchymal chondrosarcoma; giant cell tumour of bone; Ewing's sarcoma; odontogenic tumour, malignant ameloblastic odontosarcoma; ameloblastoma, malignant ameloblastic fibrosarcoma; pinealoma, malignant chordoma; glioma, malignant ependymoma; astrocytoma; protoplasmic astrocytoma; fibrillary astrocytoma; astroblastoma; glioblastoma multiforme; oligodendroglioma; oligodendroblastoma; primitive neuroectodermal; cerebellar sarcoma; ganglioneuroblastoma; neuroblastoma; retinoblastoma; olfactory neurogenic tumour; meningioma, malignant neurofibrosarcoma; neurilemmoma, malignant granular cell tumour, malignant lymphoma; Hodgkin's disease; Hodgkin's paragranuloma; malignant lymphoma, small lymphocytic malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. Preferably, the cancer is selected from bladder cancer (including non-muscle invasive bladder cancer), prostate cancer, liver cancer, renal cancer, lung cancer, breast cancer, colorectal cancer, colon cancer, rectal cancer, pancreatic cancer, brain cancer (including glioblastoma), hepatocellular cancer, lymphoma, leukaemia, gastric cancer, cervical cancer, ovarian cancer, thyroid cancer, melanoma, head and neck cancer, skin cancer and soft tissue sarcoma and/or other forms of carcinoma.

Preferably, the cancer is selected from prostate cancer, liver cancer, renal cancer, lung cancer, breast cancer, colorectal cancer, pancreatic cancer, brain cancer, hepatocellular cancer, lymphoma, leukaemia, gastric cancer, cervical cancer, ovarian cancer, thyroid cancer, melanoma, head and neck cancer, skin cancer and soft tissue sarcoma and/or other forms of carcinoma.

The tumour may be metastatic or a malignant tumour, most preferably metastatic. More preferably, the cancer is pancreatic, colorectal, prostate, ovarian cancer, most preferably the cancer is pancreatic, including pancreatic ductal adenocarcinoma (PDAC), or the cancer is metastatic pancreatic cancer, including metastatic pancreatic ductal adenocarcinoma (mPDAC), locally advanced pancreatic cancer (with or without nodal lesions), resectable pancreatic cancer, borderline resectable pancreatic cancer, checkpoint-refractory pancreatic cancer, chemotherapy-refractory pancreatic cancer or oligometastatic pancreatic cancer.

In a preferred embodiment, the invention provides an immunomodulator for use in the treatment or control of cancer comprising a primary tumour, in a patient that has undergone, or is intended to undergo tumour resection surgery and/or administration of one or more additional anticancer treatments and/or agents, comprising simultaneously, separately, or sequentially administering one or more doses of ionizing radiation selected from photon therapy or particle therapy to the patient, preferably a targeted radiotherapy such as SBRT comprising CyberKnife, GammaKnife, MRI-guided SBRT (MRldian, Elekta Unity) or such like, wherein said immunomodulator comprises non-pathogenic non-viable Mycobacterium and wherein the cancer is pancreatic cancer selected from pancreatic ductal adenocarcinoma (PDAC), pancreatic neuroendocrine tumours (PNET), metastatic pancreatic ductal adenocarcinoma (mPDAC), locally advanced pancreatic cancer “LAPC” (with or without nodal lesions), resectable pancreatic cancer, borderline resectable pancreatic cancer, checkpoint-refractory pancreatic cancer, chemotherapy-refractory pancreatic cancer or oligometastatic pancreatic cancer, preferably locally advanced pancreatic cancer or oligometastatic pancreatic cancer. The one or more additional anticancer treatments and/or agents may comprise FOLFIRINOX administration before, after or both before and after said MRI-guided SBRT and/or non-pathogenic non-viable Mycobacterium. Furthermore, such MRI-guided SBRT does not require the use of fiducial markers or seeds.

In a preferred embodiment is provided a neoadjuvant treatment for patients that have been recently diagnosed with locally advanced ductal adenocarcinoma of the pancreas over 18 years of age, with an ECOG performance status <2, planned to receive FOLFIRINOX and are, in the opinion of the Investigator, considered eligible to receive SMART (MRI-guided SBRT) if stable or responding disease, comprising administration of one or more additional anticancer treatments and/or agents, and comprising simultaneously, separately, or sequentially administering one or more doses of non-pathogenic non-viable Mycobacterium.

Radiotherapy, either alone or in combination with chemotherapy, is part of the standard treatment strategy of PDAC mostly in curative or neoadjuvant settings. To date, the recommended neo-adjuvant therapy in patients with locally advanced PDAC and European Cooperative Oncology Group (ECOG) performance status (PS) 0 or 1 is infusional 5-fluorouracil (5-FU), leucovorin/folinic acid (LV), irinotecan and oxaliplatin (mFOLFIRINOX). Immune therapies have opened new opportunities in cancer therapy. However, results of immunotherapy in PDAC have been disappointing so far, with failure of checkpoint inhibitor monotherapies (anti-CTLA4 and anti-PD-L1 monoclonal antibodies in progressive advanced PDAC. However, it remains a challenging technique based on the proximity of various radiosensitive normal structures (organs at risk, “OAR”), like the duodenum, bowel and stomach leading potentially to life threatening adverse reactions, complications such as bowel perforation. Additionally, PDAC has a high propensity to metastasize. When most patients develop distant metastatic disease, the ability for a local modality, such as surgery or radiotherapy to demonstrate meaningful improvements in OS, is diminished. Thus, this type of technology may be more appropriate in a locally advanced setting of the disease rather than in advanced metastatic disease. Despite this, ˜30% of patients with PDAC will die of predominately local disease progression. Maximizing local therapy will therefore become increasingly important for patients with PDAC and will potentially lead to better OS in an era of more effective systemic therapies. Local progression causes morbidity, which is difficult to treat. Effective local therapies can reduce symptoms and improve quality of life, both of which radiotherapy has been consistently shown to effectively accomplish and higher doses of radiotherapy have been associated with improvements in both OS and local control. While historic strategies with SBRT included tumour only, there are recently published patterns of recurrence data that suggest the possibility of higher local and regional recurrences around the vasculature associated with focal SBRT including only the tumour.

The oligometastatic state primarily refers to a stage of disease where a cancer has spread beyond the site of the primary tumour, but is not yet widely metastatic. In the ongoing phase III SABR-COMET-10 trial, 12 patients with 4 to 10 oligometastases undergo radiation preplanning before randomization to ensure that SABR can be delivered safely.

In certain embodiments, said oligometastatic pancreatic cancer is defined as being a “limited metastatic disease”, where the patients are adult patients with synchronous or metachronous limited (55 hepatic lesions and/or pulmonary lesions with a tumour volume of <9 cm per organ) metastatic pancreatic cancer after completion of at least 8 cycles of FOLFIRINOX. “Synchronous” tumours refer to cases in which the second primary cancer is diagnosed within 6 months of the primary cancer; “metachronous” tumours refer to cases in which the second primary cancer is diagnosed more than 6 months after the diagnosis of the first primary cancer or tumour.

Alternatively, the criteria for limited metastatic disease in PDAC is defined as: number of hepatic and/or pulmonary lesions ≤5, the combined diameter of all lesions per organ is less than 9 cm centimetres, WHO classification ½, no metastasis in other organs, no malignant ascites, CA 19-9<1000 U/mL and response or stable disease after FOLFIRINOX therapy.

In another embodiment of the invention, the use or method according to the invention results in, (i) increased eligibility for tumour resection, (ii) increased respectability rate across a patient cohort, (iii) increased potential for R0 or R1 tumour resection, (iv) increased potential for radical or curative surgical resection, or (v) increased potential for tumour downstaging.

In another embodiment of the invention, the one or more additional anticancer agents or modalities are to be administrated intratumorally simultaneously, separately or sequentially with the non-pathogenic non-viable whole cell Mycobacterium, optionally before and/or after said administration of one or more doses of ionizing radiation selected from photon therapy or particle therapy to the patient. For example, the non-pathogenic non-viable whole cell Mycobacterium may be delivered to the pancreatic site via intra-arterial transit using RenovoCath™ RC120 Catheter (RenovoRx). The RenovoCath™ RC120 Catheter is an endovascular multi-lumen, two-handled catheter designed to isolate variable segments of arteries supplying the target organ using two slideable, compliant balloons. Upon inflation of the proximal occlusion balloon and the distal occlusion balloon the catheter isolates the site to specifically deliver said therapeutic agents. Another suitable combination is intratumoral administration of the non-pathogenic non-viable Mycobacterium together with hafnium oxide nanoparticles, subsequently activated by radiotherapy (NBTXR3, NanoBiotix, Inc.).

Yet another suitable combination is intratumoral administration of the non-pathogenic non-viable Mycobacterium together with brachytherapy seeds such as Oncosil (Oncosil Medial, Australia). OncoSil™ is a single-use brachytherapy medical device comprising phosphorous-32 (32P) Microparticles suspended in a specially formulated diluent. The OncoSil™ suspension, containing a pre-determined activity of radiation, is injected directly into the tumour to deliver an absorbed dose of 100 Gy in 81 days. The beta radiation emitted by the OncoSil™ device travels a short distance within the tumour tissue causing direct damage to the cancer cell DNA. The microparticles remain in the tumour permanently and the volume implanted is equivalent to 8% of the tumour volume. Oncosil is delivered by a suitably qualified endoscopist via ultrasound-guided endoscopy where an echoendoscope is guided into the upper intestine. An FNA needle is then loaded through the biopsy channel of the echoendoscope and slowly guided through the gastric wall or duodenal wall into the target pancreatic tumour.

According to another embodiment of the invention, the performance status of the subject stays the same or improves during and/or after said treatment, reduction, inhibition or control of said cancer, for example, the patient may commence therapy under the invention as PS0, PS1 or PS2 and maintain said score throughout and at the end of the desired therapy or regimens, or the patient may commence therapy under the invention as PS1 and demonstrate an improved performance score throughout and/or at the end of the desired therapy or regimens, such PS0.

In another embodiment of the invention, is provided a method of treating or controlling cancer comprising a primary tumour in a patient, wherein said method comprises simultaneously, separately or sequentially administering to the subject, (i) one or more doses of ionizing radiation selected from photon therapy or particle therapy to the patient, and (ii) a non-pathogenic non-viable Mycobacterium, wherein said method results in enhanced therapeutic efficacy relative to administration of one or more doses of ionizing radiation selected from photon therapy or particle therapy, or non-pathogenic non-viable whole cell Mycobacterium alone.

In another embodiment of the invention according to said method, the patient has undergone, or is intended to undergo tumour resection surgery and/or administration of one or more additional anticancer treatments and/or agents.

In further embodiment, methods of the invention include: wherein said photon therapy comprises administration of x-rays and/or gamma rays, or wherein said particle therapy comprises administration of electrons, protons, neutrons, pions, neon ions, argon ions, silicon ions or carbon ions; or wherein said one or more doses of ionizing radiation selected from photon therapy or particle therapy is administered to the patient from an external source, such as: external-beam radiation therapy, 2-D radiation therapy, systemic 3-dimensional conformal radiation therapy (3D-CRT), rotational, helical or arc-based intensity-modulated radiation therapy (IMRT), image-guided radiation therapy (IGRT), hypofractionated cone beam radiotherapy, tomotherapy, intra-operative radiotherapy (IORT), ultra-high dose rate (FLASH) radiotherapy, stereotactic radiosurgery or stereotactic body radiation therapy (SBRT), such as via GammaKnife or CyberKnife or similar apparatuses; or wherein said one or more doses of ionizing radiation selected from photon therapy or particle therapy is administered to the patient from an internal source comprising one or more particles or nanoparticle placed within or adjacent to said cancer, which may emit free radicals, alpha-radiation, beta-radiation, x-rays or gamma rays, or combinations thereof, including hafnium oxide nanoparticles (NBTXR3, NanoBiotix, Inc), subsequently activated by radiotherapy; or wherein said one or more particles or nanoparticles comprise brachytherapy seeds containing a radioisotope, such as phosphorus-32 (such as Oncosil), or iodine-125 (American SynQor, Inc); or wherein said one or more doses of ionizing radiation selected from photon therapy or particle therapy is administered to the patient using images or real-time guidance derived from X-rays, CT, PET, ultrasound or magnetic resonance (MR), preferably via MRI-guided LINAC or SBRT, such as MRldian or Elekta Unity; or wherein said patient presents with metastases in lymph nodes and/or organs distant to the primary tumour, such as wherein said patient presents with between 1 and 10 oligometastatic tumours or lesions, or with less than 5 oligometastatic tumours or lesions, as a total between the lung and the liver; or wherein said patient does not present with metastases in any lymph nodes and/or organs distant to the primary tumour.

In further embodiment, methods of the invention comprise the adjuvant, neoadjuvant or peri-adjuvant treatment or control of cancer comprising a primary tumour, in a patient intended to undergo, or having undergone, tumour resection surgery; or wherein said one or more doses of ionizing radiation selected from photon therapy or particle therapy, comprises a maximum biologically equivalent dose at alpha/beta of 10 Gray, (BED10) of greater than about 40 Gray, or greater than about 50 Gray, or greater than about 90 Gray; or wherein said one or more doses of ionizing radiation selected from photon therapy or particle therapy is administered to the patient as a single fraction or via a fractionated regimen; or wherein said fractionated regimen comprises between 2 and 50 fractions, such as between 3 and 10 fractions, preferably between 3 and 5 fractions; or wherein the dose is between 40 and 50 Gray, administered in 5 fractions; or wherein said one or more additional anticancer treatments or agents is selected from: adoptive cell therapy, surgical (tumour resection) therapy, chemotherapy, hormonal therapy, checkpoint inhibitor therapy, small molecule therapy such as metformin, receptor kinase inhibitor therapy, hyperthermia treatment, phototherapy, radiofrequency ablation therapy (RFA), anti-angiogenic therapy, cytokine therapy, cryotherapy, biological therapy, HDAC inhibitor e.g. OKI-179, BRAF inhibitor, MEK inhibitor, EGFR inhibitor, VEGF inhibitor, P13K delta inhibitor, PARP inhibitor, mTOR inhibitor, hypomethylating agents, oncolytic virus, TLR agonist including TLR2, 3, 4, 7, 8 or 9 agonists, such as rintatolimod, or TLR 5 agonists, MRx0518 (4D Pharma), STING agonists (including MIW815 and SYNB1891), mifamurtide and cancer vaccines such as GVAX or CIMAvax, and combinations thereof; or wherein the cancer is selected from bladder cancer (including non-muscle invasive bladder cancer or urothelial cancer), prostate cancer, liver cancer, renal cancer, lung cancer, breast cancer, colorectal cancer, colon cancer, rectal cancer, pancreatic cancer, brain cancer (including glioblastoma), hepatocellular cancer, lymphoma, leukaemia, gastric cancer, cervical cancer, ovarian cancer, thyroid cancer, melanoma, head and neck cancer, skin cancer and soft tissue sarcoma and/or osteosarcoma, including pancreatic ductal adenocarcinoma (PDAC) and pancreatic neuroendocrine tumours (PNET), optionally wherein said cancer is metastatic; or preferably, wherein the cancer is pancreatic cancer is selected from: locally advanced pancreatic cancer (with or without nodal lesions), resectable pancreatic cancer, borderline resectable pancreatic cancer, checkpoint-refractory pancreatic cancer, chemotherapy-refractory pancreatic cancer, oligometastatic pancreatic cancer, pancreatic ductal adenocarcinoma (PDAC), and metastatic pancreatic ductal adenocarcinoma (mPDAC);

Alternatively, said method applied to a cancer clinically defined by a TNM staging criteria, in which the patient has a primary tumour (T) of T1 to T4, and/or or a Node designation of NO, N1 or N2, or wherein said cancer is clinically defined as being Stage I, Stage II or Stage III or Stage IV, optionally wherein the patient has no evidence of metastasis (M0); or, wherein the patient has undergone, or is intended to undergo, tumour resection surgery, optionally wherein the tumour resection surgery further comprises lymph node resection and optionally metastatic tumour resection when metastatic disease is present, such as metastatic pancreatic ductal adenocarcinoma (mPDAC).

In an embodiment of the invention, the use, combinations and methods disclosed herein result in a clinically relevant improvement in one or more markers of disease status and progression selected from one or more of the following: (i): overall survival, (ii): progression-free survival, (iii): overall response rate, (iv): reduction in metastatic disease, (v): circulating levels of tumour antigens such as carbohydrate antigen 19.9 (CA19.9), carcinoembryonic antigen (CEA), prostate-specific antigen (PSA) or others depending on tumour, or enhanced CXCL10 expression, (vii) nutritional status (weight, appetite, serum albumin) and a reduction in loss of lean body mass i.e. prevention or reduction in cachexia (viii): systemic immune-inflammation index (SII) or systemic inflammation score (SIS), (ix): pain control or analgesic use, or (x): CRP/albumin ratio or prognostic nutritional index (PNI), or neutrophil/lymphocyte ratio (NLR), or (xi) improved Quality of Life, or (xii), a reduction or elimination in ctDNA, preferably as assessed at 12 months post-surgery or end of photon therapy or particle therapy.

In some embodiments, the one or more markers of disease status and progression selected from the above list may be measured for monitoring of the treatment, reduction, inhibition or control protocols of the present invention. In some preferred embodiments, the one or more biomarkers may include any one or more of: prostate-specific antigen (PSA); carcinoembryonic antigen (CEA); prognostic nutritional index (PNI); systemic immune-inflammation index (SII); or neutrophil/lymphocyte ratio (NLR) and systemic inflammation score (SIS).

The invention is further described with reference to the following non-limiting Examples.

### Example 1

A phase I/II study has been developed to investigate the safety and efficacy of the addition of IMM-101 (Heat-Killed Whole Cell M. obuense NCT13365) to standard stereotactic radiotherapy (SBRT, CyberKnife), in locally advanced pancreatic cancer patients. (LAPC-2 trial—Dutch Trial Register entry: NL7578) https://www.trialregister.nl/trial/7578

Summary—this phase I/II study consists of 2 subsequent study parts. Phase I part involves investigating the safety of combining IMM-101 administration with SBRT in 20 patients with locally advanced pancreatic cancer who have completed at least 4 cycles of FOLFIRINOX chemotherapy. Once deemed safe and feasible (defined as max 6 out of 20 patients experiencing a grade 4/5 toxicity related to the IMM-101 intervention), inclusion was continued into phase II with an additional 18 patients in order to be able to study efficacy of combining IMM-101 treatment with SBRT based on a 20% improvement of 1-year disease free survival. Secondary endpoints are overall survival, time to locoregional progression, time to distant metastasis, feasibility, safety/toxicity, resection rate, tumour specific immune-responses and quality of life/sleep.

Target size—38 patients

Inclusion criteria—Histologically confirmed pancreatic cancer, as indicated by a definite cytology report; tumour considered locally advanced after diagnostic work-up including CT-imaging, using the DPCG criteria for locally advanced disease and diagnostic laparoscopy; Age >18 years and <75 years; WHO (ECOG) performance status of 0 or 1; ASA classification I or II; No evidence of metastatic disease; Largest tumour size <7 cm×7 cm×7 cm; normal renal function (Creatinine ≥30 ml/min); Normal liver tests (bilirubin<1.5 times normal*; ALAT/ASAT<5 times normal); Normal bone marrow function (WBC>3.0×10e9/L, platelets >100×10e9/L and hemoglobin >5.6 mmol/1).

Hypothesis—approximately 30-40% of patients with pancreatic cancer present with locally advanced pancreatic cancer. Patients with locally advanced pancreatic cancer cannot be surgically resected but at the same time have no clinically detectable distant metastasis. Current treatment regimens consist of the use of neoadjuvant chemotherapy such as FOLFIRINOX, followed by stereotactic body radiation therapy. Despite slow improvements in patient outcomes, this strategy results in only approximately a third of patients being surgically resectable and an overall survival of only 10-12 months. Recently, improved understanding in the field of tumor immunology has led to progress and breakthroughs in cancer immunotherapeutic strategies. One such therapeutic strategy is immunotherapy using modulators of the immune system. Radiation therapy can act as an in-situ vaccine, increasing the expression of cell surface receptors and tumor antigen presentation and can even produce anti-tumor cytotoxic T cell response. However, optimal anti-tumor response requires an intact host's immune system and without amplification, the anti-tumor immunity arising from radiation therapy is likely to be limited. It is hypothesized that the combination of boosting of the body's immune responses in the presence of an increased exposure to tumor antigen will provide sufficient induction of the immune system to counter further tumor growth. IMM-101, through its activation and maturation of antigen presenting cells, and especially dendritic cells, can aid in the antigen processing and T-cell cross priming, processes that are deficient in the setting of advanced pancreatic cancer. IMM-101 immunotherapy thereby has the potential to optimize the immunogenic anti-tumor effect of radiation therapy.

Interventions—Six intradermal injections of IMM-101 (a vaccine adjuvant containing Heat-Killed Whole Cell Mycobacterium obuense) beginning 2 weeks prior to stereotactic body radiation therapy. Between the third and fourth injection will be a four-week break. Administration of IMM-101 will be performed at week 0, 2, 4, 8, 10 and 12.

### Primary Outcome

Phase I—the main endpoint of the first inclusion phase is to determine safety/toxicity of IMM-101 administration in LAPC patients undergoing SBRT. Safety/toxicity of the IMM-101 intervention will be determined according to CTCAE version 5.0. All grade 4 and 5 events related to the administration of the IMM-101 product will be considered events for this endpoint.

Phase II—The main endpoint of the second inclusion phase is to asses efficacy of IMM-101 therapy in combination with SBRT in LAPC patients. Efficacy will be determined using 1-year PFS rates. PFS is defined as survival without locoregional progressive disease, the occurrence of distant metastases, the occurrence of second or recurrent pancreatic cancer from the date of inclusion. All included patients (i.e. 38 patients) will be analyzed for this endpoint.

### Secondary Outcome

For all patients the following secondary endpoints will be determined: Overall survival; Time to locoregional progression, defined as the period of time without locoregional progression after inclusion; Time to distant metastasis, defined as the period of time without distant metastases after inclusion; Radiological response rate after IMM-101 and SBRT using RECIST criteria (version 1.1); Resection rate defined as the percentage of included patients that underwent a curative-intent resection; Feasibility of receiving IMM-101 treatment and performing follow-up. Defined as feasibility of treatment procedures in order to be able to administer IMM-101 to patients and follow up these patients (e.g. ability to collect extra blood samples at designated time points); Safety/Toxicity according to CTCAE 5.0; Tumor-specific immune responses; Quality of sleep and sleep duration; Quality of life;

Sponsors—Erasmus MC, Surgery department

### Time Points

Screening, baseline, week 2/4/8/10/12/14 and 26. Following study week 26 standard FU will be performed at month 9/12/15/18/21/24/36/42/48/54 and 60.

At the time of writing, this LAPC-2 study has completed recruitment and all patients have completed their initial 14-week visit; the time point at which tumour respectability is evaluated. Five patients continue on study and will receive study medication until week 26. In LAPC-1 trial (SBRT alone in treating LAPC patients; 10.1016/j.eclinm.2019.10.013), 12% of the patients treated with SRBT following neoadjuvant chemotherapy subsequently underwent tumour resection at the end of the treatment period. Median progression free (PFS) and overall survival (OS) were 11 months and 15 months respectively while the 1-year survival rate was 64%.

To date, the combination of SBRT with IMM-101 has provided an overall acceptable safety profile with no SAE reported as related to IMM-101 and most of the AEs reported as related to IMM-101 being CTCAE grade 1 or 2 (mainly local erythema at the site of injection, fever, and some fatigue) as previously reported. Most of the SAEs reported in the LAPC-2 study were related to disease progression and very few reported as potentially linked to SBRT itself.

Although the study remains ongoing, the rate of respectability observed in this study to date is higher than the 12% rate initially reported in LAPC-1 (DOI: 10.1016/j.eclinm.2019.10.013). The other endpoints will not be determined until study completion.

### Example 2

A study has been developed to investigate the safety and efficacy of the addition of IMM-101 (Heat-Killed Whole Cell M. obuense NCT13365) to standard stereotactic radiotherapy (SBRT, CyberKnife), in patients with limited MEtastatic PANcreatic Cancer (MEPANC-1 trial—Dutch Trial Register entry: NL8819) https://www.trialregister.nl/trial/8819

Summary—approximately 40% to 50% of patients diagnosed with pancreatic cancer present with metastatic disease. In addition, 80% of patients with resected pancreatic ductal adenocarcinoma (PDAC) develop recurrent disease due to its aggressive nature and its tendency to develop early metastases. Around 25% of patients with recurrence develop liver only metastases. Lung-only recurrence is seen in 14.7% of the patients. All these patients including those who develop metastases after resection are currently treated with palliative chemotherapeutic drugs, regardless of their pattern of metastases. Median overall survival in these metastatic patients treated with FOLFIRINOX, the most effective chemotherapeutic regimen is shorter than 11 months with a 1-year progression free survival of 12%. Patients with limited hepatic and/or pulmonary metastases could potentially benefit from additional multimodality treatment after standard palliative treatment. This treatment could be effective for patients with few metastases to limited organ sites, also known as oligometastatic disease. Recently, a new definition of oligometastatic disease in PDAC was proposed, which includes anatomical and biological criteria. We define limited metastatic disease as 55 metastases in the liver and/or lungs with a total tumour size per organ of <9 cm. The concept of limited metastatic disease creates extra treatment approaches for each patient's individual metastatic state. The addition of Stereotactic Body Radiation (SBRT) with immunotherapy (IMM-101) after FOLFIRINOX will harness the immune system in a synergistic approach. SBRT can act as an in-situ vaccine, increasing the expression of cell surface receptors and tumour antigen presentation and can even produce anti-tumour cytotoxic T cell response. In addition, IMM-101 (suspension of heat-killed whole cell Mycobacterium obuense) activates and maturates antigen presenting cells. Especially dendritic cells can aid in the antigen processing and T-cell cross priming, processes that are deficient in the setting of metastatic pancreatic cancer. IMM-101 immunotherapy thereby has the potential to optimize the immunogenic anti-tumour effect of radiation therapy. The combination of boosting the immune responses with immunotherapy in the presence of an increased exposure to tumour antigen will provide sufficient induction of the immune system to counter further metastatic burden. We hypothesize that treatment of IMM-101 combined with SBRT in patients with limited metastatic hepatic or pulmonary disease of PDAC induces a durable local and systemic anti-tumour immune response to obtain disease control. This IMM-101 and SBRT protocol will start 4 weeks after standard chemotherapy treatment (FOLFIRINOX).

Target size—100 patients

Inclusion criteria—histologically confirmed metastatic pancreatic cancer, as indicated by a definite cytology/histology report; 55 hepatic and/or pulmonary metastases in total; the combined diameter of all liver metastases AND the primary tumour or local recurrence in the pancreas is <9 cm; the combined diameter of all pulmonary metastases is <9 cm; CA 19-9<1000 IU/mL after completion of chemotherapy; Age >18 years and <75 years; WHO (ECOG) performance status of 0 to 2; tumour volume of the primary tumour <7 cm×7 cm×7 cm; each diameter must not exceed 7 cm; adequate renal function (Creatinine >30 ml/min); adequate liver tests (bilirubin <1.5 times normal; ALAT/ASAT <5 times normal); adequate bone marrow function (WBC >3.0×109/L, platelets >100×10\hat{ }9/L and hemoglobin >5.6 mmol/I); effective contraceptive method; written informed consent; patients who did not complete at least 8 cycles of chemotherapy due to severe toxicity, will be included in the expansion cohort.

Exclusion criteria—metastasis in other organs than the lung and liver; histopathologically proven extra regional lymph node metastasis; malignant ascites; liver function insufficient to tolerate the prescribed dose of radiotherapy; Child-Pugh Classification grade B/C; lung function insufficient to tolerate the prescribed dose of radiotherapy; diffuse liver metastasis pattern on CT scan; current or previous treatment with immunotherapeutic drugs; second primary malignancy except in situ carcinoma of the cervix, adequately treated non-melanoma skin cancer, or other malignancy treated at least 5 years previously to diagnosis of pancreatic cancer and without evidence of recurrence.

Hypothesis—the addition of Stereotactic Body Radiation (SBRT) with immunotherapy (IMM-101) after FOLFIRINOX will harness the immune system in a synergistic approach. SBRT can act as an in-situ vaccine, increasing the expression of cell surface receptors and tumour antigen presentation and can even produce anti-tumour cytotoxic T cell response. In addition, IMM-101 (suspension of heat-killed whole cell Mycobacterium obuense NCTC 13365) activates and maturates antigen presenting cells. Especially dendritic cells can aid in the antigen processing and T-cell cross priming, processes that are deficient in the setting of metastatic pancreatic cancer. IMM-101 immunotherapy thereby has the potential to optimize the immunogenic anti-tumour effect of radiation therapy. The combination of boosting the immune responses with immunotherapy in the presence of an increased exposure to tumour antigen will provide sufficient induction of the immune system to counter further metastatic burden. We hypothesize that treatment of IMM-101 combined with SBRT in patients with limited metastatic hepatic or pulmonary disease of PDAC induces a durable local and systemic anti-tumour immune response to obtain disease control. This IMM-101 and SBRT protocol will start 4 weeks after standard chemotherapy treatment (FOLFIRINOX).

Interventions—IMM-101 immunotherapy and stereotactic radiotherapy (SBRT)

Primary outcome—The main goal of the safety run-in is to determine the safety/toxicity profile of IMM-101 vaccination in combination with SBRT in patients with limited meta- or synchronous metastatic disease liver and/or lung metastatic disease from pancreatic cancer.

Secondary outcome—Overall survival calculated from the start of FOLFIRINOX (OS1); Overall survival calculated from start of IMM-101 (OS2); Progression-free survival calculated from the start of IMM-101 (PFS 2) at 12-month to the date of progressive disease of the primary tumour, locoregional recurrence, progression of previously treated lungs and/or liver metastases, the occurrence of new metastases, or death. All included patients will be analysed for this endpoint; Quality of Life; Radiological response rate after IMM-101 and SBRT using RECIST criteria (version 1.1); Immunological effects: effect of IMM-101 and SBRT on circulating immune cells; Effect on tumour markers, CA19.9 and CEA.

Sponsors—Erasmus MC

Time points—Week 0, 2, 4, 8, 10, 12, 16, 20, 24, 28, 32, 36, 40, 44, 48 and 52

This study, at the time of writing, has commenced recruiting patients.

### Example 3

A Phase I/II, open label, non-randomized, Study to Evaluate the Safety and Efficacy of IMM-101 in Combination with FOLFIRINOX, Stereotactic MRI-Guided Adaptive Radiotherapy (SMART) and a checkpoint inhibitor in Patients with Locally advanced Pancreatic Ductal Adenocarcinoma (PDAC). This is a neoadjuvant treatment for patients that have been recently diagnosed with locally advanced ductal adenocarcinoma of the pancreas over 18 years of age, with an ECOG performance status <2, planned to receive FOLFIRINOX and are, in the opinion of the Investigator, considered eligible to receive SMART if stable or responding disease.

Study Duration—up to 38 patients will be treated for a duration of 12 months.

Inclusion Criteria are: Male and female 18 years old; histologically or cytologically confirmed locally advanced adenocarcinoma of the pancreas; ECOG Performance status 0 to 1 (<2); at least 3 months life expectancy; lesion with clear margin but no option for surgical resection at time of diagnosis or radiation with curative intent; participants must have measurable disease, defined as a lesion that can be accurately measured in at least one dimension (longest diameter to be recorded for non-nodal lesions and short axis for nodal lesions) as ≥20 mm with conventional techniques or as 10 mm with spiral CT scan, MRI, (Radiographically assessable disease according to RECIST V1.1 criteria); patients eligible to receive FOLFIRINOX as a neoadjuvant regimen; no evidence of distant metastasis; pancreatic tumour size ≤7 cm in the longest axial dimension; ability to understand and follow the breathing instructions involved in the respiratory gating procedure; participants must have normal organ and marrow function as defined as a. absolute neutrophil count ≥1,500/mcL; b. adequate lymphocyte count; c. platelets 100,000/mcL; d. total bilirubin within normal institutional limits; AST(SGOT)/ALT(SGPT)≤2.5×institutional upper limit of normal; creatinine within normal institutional limits OR Creatinine clearance ≥60 mL/min/1.73 m2 for participants with creatinine levels above institutional normal; females of childbearing potential (FCBP) must have a negative highly sensitive serum pregnancy test within 7 days of the first administration of study drug.

IMM-101 is to be given via intradermal injection into the skin overlying the deltoid muscle, with the arm being alternated between each dose. IMM 101 will be administered in the morning and prior to chemotherapy for the days where they are administered together. IMM-101 will be administered at a dose 1.0 mg (0.1 mL) intra dermally, the volume of 0.1 mL containing 10 mg/mL of IMM-101 will be injected at week 0, 2, 4, 8, 10 and 12.

CPI is administered according to the manufacturer's instructions/sMPC, chosen from nivolumab, durvalumab, avelumab, pemobrolizumab, or atezelizumab, or as chosen by the Investigator.

The primary objective is: to evaluate the safety and tolerability of the combination of IMM-101 with mFOLFIRINOX, a Checkpoint inhibitor (CPI) and SMART in locally advanced PDAC patients by examining the profile of adverse events experienced (changes in clinical status and in laboratory parameters, including local tolerability at the injection site of IMM-101).

The secondary objectives are: To evaluate the efficacy of IMM-101 combined with mFOLFIRINOX, a CPI and SMART by evaluating the 1-year progression free survival rate (PFS rate); to evaluate the Overall Survival at 1 year in locally advanced PDAC patients over 18 years of age or older at enrollment receiving IMM-101, mFOLFIRINOX, a CPI and SMART; to evaluate the Overall Survival at 5 years in locally advanced PDAC patients over 18 years of age or older at enrollment receiving IMM-101, mFOLFIRINOX, a CPI and SMART; time to locoregional progression, defined as the period without locoregional progression from consent to signs of locoregional progression as determined via imaging (CT-scan or MRI); time to distant metastasis, defined as the period without distant metastases after consent to images of metastases as via imaging (CT-scan or MRI); Radiological response rate after IMM-101, mFOLFIRINOX, a CPI and SMART using RECIST criteria (version 1.1); resection rate defined as the percentage of patients included into the study that undergo a curative resection as determined after Week 14, visit to determine the eligibility and the intent to resect.

The exploratory objectives are: Quality of Life Questionnaire C30 (QLQ-C30) at baseline, 1, 2, 3 months; The General Sleep Disturbances Questionnaire will be used to determine the quality of sleep, sleep efficiency and sleep duration; Sample collections for biological and immunological markers (tumor specific and non-tumor specific); collection of tumour block extracted and fixed during resection of pancreatic tumour for further analysis.

This study will recruit patients over 18 years of age recently diagnosed with locally advanced PDAC, with with an ECOG performance status <2 planned to receive mFOLFIRINOX as neoadjuvant therapy and considered suitable for SBRT/or SMART and treated with an intent to resect as an outcome for their locally advanced disease.

Once consented, patients will undergo screening tests. Qualifying patients will receive IMM-101 combined to mFOLFIRINOX, a CPI and SMART (8 Gy per radiation dose on 5 alternate days) Chemotherapy and CPI to be administered according to ‘standard of care’.

Vitamin D levels will be measured in all patients at screening, patients with levels lower than the normal range will be supplemented to normalise their levels while receiving the investigational product. Their vitamin levels will be monitored according to UK guidelines.

Additional samples will be collected to evaluate immunology responses. And biopsy blocks will be collected during resection procedure for further analyses.

Patients will undergo at screening and week 12 a PET-scan for a better disease evaluation (more specifically for loco-regional disease and response to treatment, allowing to distinguish between metabolic active and no metabolic active lesions).

Patients on study will receive the investigational regimen until they are evaluated for pancreatic tumour resectability at Week 14. Based on the outcome, patients may: 1. have a tumour resection, 2. patients that do not qualify for resection will be transitioned back to standard of care FOLFIRINOX until disease progression, withdraw or death. Once patients have exited study—resected or continued to mFOLFIRINOX—they will be followed quarterly via phone for 5 years total to capture their disease status (remission, progression and/or death).

Patients will receive a first dose of IMM-101 at a volume of 0.1 mL containing 10 mg/mL administered intra dermally followed two weeks later by the first cycle of mFOLFIRINOX and the CPI. mFOLFIRINOX will be administered to the patients every 2 weeks for 6 cycles as follows: oxaliplatin 85 mg/m2 infused over 120 min, immediately followed by folinic acid (Leucovorin) 400 mg/m2 infused over 120 min with the addition, after 30 minutes of irinotecan at a dose of 135 mg/m2 infused over 90 min, followed by 5 FU 300 mg/m2 i.v. bolus, followed by 2400 mg/m2 continuous infusion for 46 h (25% reduction in bolus 5 FU and irinotecan doses). At each time the patients are to receive IMM-101 and mFOLFIRINOX, IMM-101 will be administered first, followed an hour later by mFOLFIRINOX and the CPI. The CPI will be administered according to is package insert recommendations. Two Four weeks after the completion of the first 6 cycles of FOLFIRINOX in combination with CPI and IMM-101, patients will receive SMART. Patients will receive a total nominal dose of 40 Gray (Gy) in 5 fractions of 8 Gray each over 5 alternates days.

Patients will be evaluated for safety and efficacy according to the schedule of events to determine their performance on investigational study product. Various laboratory, clinical, imaging evaluations and quality of life assessments will be performed throughout the study, from the screening visit to the last follow-up study visit.

1-99. (canceled)

100. A method of treating or controlling cancer comprising a primary tumour in a patient, wherein said method comprises simultaneously, separately or sequentially administering to the subject, (i) one or more doses of ionizing radiation selected from photon therapy or particle therapy to the patient, and (ii) a non-pathogenic non-viable Mycobacterium, wherein said method results in enhanced therapeutic efficacy relative to administration of one or more doses of ionizing radiation selected from photon therapy or particle therapy, or non-pathogenic non-viable whole cell Mycobacterium alone.

101. The method according to claim 100, wherein said patient has undergone, or is intended to undergo tumour resection surgery and/or administration of one or more additional anticancer treatments and/or agents.

102. The method according to claim 100, wherein said photon therapy comprises administration of x-rays and/or gamma rays.

103. The method according to claim 100, wherein said particle therapy comprises administration of electrons, protons, neutrons, pions, neon ions, argon ions, silicon ions or carbon ions.

104. The method according to claim 100, wherein said one or more doses of ionizing radiation selected from photon therapy or particle therapy is administered to the patient from an external source.

105. The method according to claim 104, wherein said external source of photon therapy is selected from external-beam radiation therapy, 2-D radiation therapy, systemic 3-dimensional conformal radiation therapy (3D-CRT), rotational, helical or arc-based intensity-modulated radiation therapy (IMRT), image-guided radiation therapy (IGRT), hypofractionated cone beam radiotherapy, tomotherapy, intra-operative radiotherapy (IORT), ultra-high dose rate (FLASH) radiotherapy, stereotactic radiosurgery or stereotactic body radiation therapy (SBRT).

106. The method according to claim 100, wherein said one or more doses of ionizing radiation selected from photon therapy or particle therapy is administered to the patient from an internal source comprising one or more particles or nanoparticle placed within or adjacent to said cancer.

107. The method according to claim 106, wherein said one or more particles or nanoparticles placed within or adjacent to said cancer emit free radicals, alpha-radiation, beta-radiation, x-rays or gamma rays, or combinations thereof.

108. The method according to claim 107, wherein said one or more particles or nanoparticles comprise brachytherapy seeds containing a radioisotope.

109. The method according to claim 100, wherein said one or more doses of ionizing radiation selected from photon therapy or particle therapy is administered to the patient using images or real-time guidance derived from X-rays, CT, PET, ultrasound or magnetic resonance (MR).

110. The method according to claim 100, wherein said patient presents with metastases in lymph nodes and/or organs distant to the primary tumour, wherein said patient presents with between 1 and 10 oligometastatic tumours or lesions.

111. The method according to claim 100, wherein said patient presents with less than 5 oligometastatic tumours or lesions, as a total between the lung and the liver.

112. The method according to claim 100, wherein said patient does not present with metastases in any lymph nodes and/or organs distant to the primary tumour.

113. The method according to claim 100, wherein said method comprises adjuvant, neoadjuvant or peri-adjuvant treatment or control of cancer comprising a primary tumour, in a patient intended to undergo, or having undergone, tumour resection surgery.

114. The method according to claim 100, wherein said one or more doses of ionizing radiation selected from photon therapy or particle therapy, comprises a maximum biologically equivalent dose at alpha/beta of 10 Gray, (BED10) of greater than about 40 Gray, or greater than about 50 Gray, or greater than about 90 Gray.

115. The method according to claim 100, wherein said one or more doses of ionizing radiation selected from photon therapy or particle therapy is administered to the patient as a single fraction or via a fractionated regimen.

116. The method according to claim 100, wherein the dose is between 40 and 50 Gray, administered in 5 fractions on sequential or alternate days.

117. The method according to claim 101, wherein said one or more additional anticancer treatments or agents is selected from adoptive cell therapy, surgical tumour resection therapy, chemotherapy, hormonal therapy, checkpoint inhibitor therapy, small molecule therapy, receptor kinase inhibitor therapy, hyperthermia treatment, phototherapy, radiofrequency ablation therapy (RFA), anti-angiogenic therapy, cytokine therapy, cryotherapy, biological therapy, HDAC inhibitor, BRAF inhibitor, MEK inhibitor, EGFR inhibitor, VEGF inhibitor, P13K delta inhibitor, PARP inhibitor, mTOR inhibitor, hypomethylating agents, oncolytic virus, TLR agonists, STING agonists, mifamurtide, cancer vaccines, and combinations thereof.

118. The method according to claim 100, wherein the cancer is selected from bladder cancer, prostate cancer, liver cancer, renal cancer, lung cancer, breast cancer, colorectal cancer, colon cancer, rectal cancer, pancreatic cancer, brain cancer, hepatocellular cancer, lymphoma, leukaemia, gastric cancer, cervical cancer, ovarian cancer, thyroid cancer, melanoma, head and neck cancer, skin cancer and soft tissue sarcoma and/or osteosarcoma, pancreatic ductal adenocarcinoma (PDAC) and pancreatic neuroendocrine tumours (PNET), optionally wherein said cancer is metastatic.
